(19) United States
(12) Patent Application Publication
(10) Pub. No.: US 2021/0277131 A1
(43) Pub. Date: Sep. 9, 2021

(54) TREATMENT EMPLOYING ANTI-IL-l3R ANTIBODY OR BINDING FRAGMENT THEREOF

(71) Applicants: Aslan Pharmaceuticals PTE LTD, Singapore (SG); CSL LIMITED, Parkville (AU)

(72) Inventor: Alison WARD, Singapore (SG)

(21) Appl. No.: 17/272,243

(22) PCT Filed: Mar. 26, 2020
(86) PCT No.: PCT/SG2020/050170

§ 371 Date: Feb. 26, 2021

(30) Foreign Application Priority Data
Mar. 26, 2019 (SG) 10201902713S
Jun. 3, 2019 (SG) 10201905063R
Aug. 16, 2019 (SG) 10201907597W

(51) Int. Cl. C07K 16/28; A61P 29/00

(52) U.S. Cl. CPC C07K 16/2866; A61K 2039/545; C07K 2317/94; A61P 29/00

(57) ABSTRACT

The present disclosure provides a method of treatment comprising inhibiting IL-13R with an antibody or binding fragment thereof specific for the receptor with a VH sequence of SEQ ID NO: 51 or a sequence at least 95% identical thereto, and VL sequence of SEQ ID NO: 53 or a sequence at least 95% identical thereto, wherein said antibody or binding fragment is administered at a dose in the range 600 mg to 900 mg at least once each month, in particular less than twice a month.

The present disclosure relates to a method of treatment comprising inhibiting IL-13R with an antibody or binding fragment thereof specific for the receptor, for example for the treatment of a patient having an inflammatory disorder or autoimmune disease. The disclosure also extends to formulations of the anti-IL13R antibody or binding fragment described herein and their use in the disclosed method of treatment.

### BACKGROUND

IL-13 has been associated with various conditions including, but not limited to, various respiratory and allergy-mediated disorders, fibrosis, scleroderma, inflammatory bowel disease and certain cancers; see, e.g., Wynn, T. A., 2003 Annu. Rev. Immunol. 21:425-456; Terabe et al, 2000 Nat Immunol. 1 (6): 515-520; Fuss et al, 2004 J. Clin. Invest. 113 (10): 1490-1497; Simms et al, 2002 Curr. Opin. Rheumatol. 14 (6):717-722; and Hasegawa et al, 1997 J. Rheumatol. 24 (2): 328-332.

Thus, IL-13 is an attractive target for the treatment of such diseases.

One possible way to inhibit the activity of IL-13 is to interfere with the binding of IL-13 to its receptor IL-13R, for example by using an antibody specific to IL-13R, such as an antibody specific to IL-13Rα1. An effective antibody antagonist to IL-13Rα1 may also interfere with the binding of IL-13 and prevent heterodimerization of IL-4Rα and IL-13Rα1. Such an antibody will inhibit signaling of both IL-13 and IL-4 through the type II receptor (formed by IL-13Rα1 and IL-4Rα) while sparing IL-4 signalling through the type I receptor. Signalling through the type I receptor is essential in the induction phase of the immune response during which Th2 cells differentiate. T cells do not express IL-13Rα1 so the type II receptor plays no role in Th2 differentiation. Hence, an IL-13Rα1 antibody may not affect the overall Th1/Th2 balance. Signalling through the type II IL-4/IL-13 receptor is critical during the effector-A-stage of the immune response during established allergic inflammation. Thus, blockade of the type II receptor should have a beneficial effect on many of the symptoms of asthma and other IL-13R-mediated conditions and may be an effective disease modifying agent.

Antibodies against IL-13Rα1 (both monoclonal and polyclonal) have been described in the art; see, eg, WO 97/15663, WO 03/80675; WO 03/46009; WO 06/072564; Gauchat et al, 1998 Eur. J. Immunol. 28:4286-4298; Gauchat et al, 2000 Eur. J. Immunol. 30:3157-3164; Clement et al, 1997 Cytokine 9(11):959 (Meeting Abstract); Ogata et al, 1998 J. Biol. Chem. 273:9864-9871; Graber et al, 1998 Eur. J. Immunol. 28:4286-4298; C. Vermot-Desroches et al, 2000 Tissue Antigens 5(Supp. 1):52-53 (Meeting Abstract); Poudrier et al, 2000 Eur. J. Immunol. 30:3157-3164; Akaiwa et al, 2001 Cytokine 13:75-84; Cancino-Diaz et al, 2002 J. Invest. Dermatol. 119:1114-1120; and Krause et al, 2006 Mol. Immunol. 43:1799-1807.

One particularly promising anti-IL-13Rα1 antibody is described in WO2008/060813 as antibody 10G5-6. 10G5-6 as an IgG4 with a hinge stabilising serine to proline mutation (S241P Kabat numbering) is known as ASLAN004. ASLAN004 has been shown to bind to human IL-13Rα1 with a high affinity (for example Kd may be 500 pM). ASLAN004 was shown to effectively antagonise IL-13 function through inhibiting the binding of IL-13 to its receptor IL-13Rα1 and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells.

However, an optimised dosage regimen for IL-13R antibodies, such as ASLAN004 is required in order to maximise therapeutic effect and/or minimise adverse effects.

### SUMMARY OF THE DISCLOSURE

The present disclosure is summarised by the following paragraphs:
1. A method of treatment comprising inhibiting IL-13R with an antibody or binding fragment thereof specific for the receptor,
   wherein each dose of the anti-IL13R antibody or binding fragment thereof is in the range of about 1 mg/kg to about 15 mg/kg (about 50 to 1000 mg, such as 60 to about 900 mg), for example about 3 mg/kg to about 15 mg/kg (about 200 to about 900 mg), about 3 mg/kg to 10 mg/kg (about 200 to about 600 mg), or about 10 mg/kg to about 15 mg/kg (about 600 to about 900 mg), in particular about 3 mg/kg to about 10 mg/kg (about 200 to about 600 mg); and wherein each dose is administered parenterally (for example intravenously) at least once a month, for example once every 4 weeks, once every 3 weeks, once every 2 weeks, or once a week, in particular only once a month.
2. The method according to paragraph 1, wherein each dose is 3 mg/kg to about 15 mg/kg, such as 3, 4, 5, 6, 7, 8, 9, 10, 11, 12, 13, 14 or 15 mg/kg.
3. The method according to paragraphs 1 or 2, wherein each dose is in the range of about 3 mg/kg to about 10 mg/kg, such as 3, 4, 5, 6, 7, 8, 9 or 10 mg/kg.
4. The method according to paragraphs 1 or 2, wherein each dose is in the range of about 10 mg to about 15 mg/kg, such as 10, 11, 12, 13, 14 or 15 mg/kg.
5. The method according to paragraphs 1 or 2, wherein each dose is about 200 to 900 mg, such as 200, 300, 400, 500, 600, 700, 800 or 900 mg.
6. The method according to paragraphs 1 or 2, wherein each dose is about 200 to 600 mg, such as 200, 250, 300, 350, 400, 450, 500, 550 or 600 mg.
7. The method according to paragraphs 1 or 2, wherein each dose is about 600 mg to about 900 mg, such as 600, 650, 700, 750, 800, 850 or 900 mg.
8. The method according to paragraphs 1 or 2, wherein each dose is 200 mg.
9. The method according to paragraphs 1 or 2, wherein each dose is 600 mg.
10. The method according to any one of paragraphs 1 to 9, wherein each dose is administered once every 3 weeks.
11. The method according to any one of paragraphs 1 to 9, wherein each dose is administered once a month.
12. The method according to any one of paragraphs 1 to 11, wherein each dose is 200 mg and is administered once every 3 weeks.
13. The method according to any one of paragraphs 1 to 12, wherein each dose is in the range 600 mg to 900 mg and is administered only once each month.
14. The method according to any one of paragraphs 1 to 13, wherein each dose is 600 mg and is administered once a month.
15. The method according to any one of paragraphs 1 to 14, where in the antibody or binding fragment thereof is for subcutaneous administration (for example administered subcutaneously).
16. The method according to any one of paragraphs 1 to 14, wherein the antibody or binding fragment is for intramuscular administration (for example administered intramuscularly).
17. The method according to any one of paragraphs 1 to 16, wherein the antibody or binding fragment thereof is provided in a depot formulation, for example for slow release.
18. The method according to any one of paragraphs 1 to 14, wherein the antibody or binding fragment is for intravenous administration (administered intravenously)
19. The method according to any one of paragraphs 1 to 18, wherein the anti-IL-13R antibody or binding fragment thereof is an anti-IL13Rα1 antibody.
20. The method according to any one of paragraphs 1 to 19, wherein the anti-IL-13R antibody or binding fragment thereof binds to the epitope FFYQ.
21. The method according to any one of paragraphs 1 to 20, wherein the anti-IL-13R antibody or binding fragment thereof comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10.
22. The method according to any one of paragraphs 1 to 21, wherein the anti-IL-13R antibody or binding fragment thereof comprises a VH domain comprising an amino acid sequence shown in SEQ ID NO: 51 or a sequence at least 95% identical thereto.
23. The method according to any one of paragraphs 1 to 22, wherein the anti-IL-13R antibody or binding fragment thereof comprises a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.
24. The method according to any one of paragraphs 1 to 23, wherein the anti-IL-13R antibody or binding fragment thereof comprises a VL domain comprising an amino acid sequence shown in SEQ ID NO: 53 or a sequence at least 95% identical thereto.
25. The method according to any one of paragraphs 1 to 24, wherein the antibody or binding fragment thereof comprises a VH domain comprising an amino acid sequence shown in SEQ ID NO: 51 or a sequence at least 95% identical thereto, and a VL domain comprising an amino acid sequence shown in SEQ ID NO: 53 or a sequence at least 95% identical thereto.
26. The method according to any one of paragraphs 1 to 25, wherein the anti-IL-13R antibody or binding fragment thereof is administered as a pharmaceutical formulation, for example a parenteral formulation.
27. The method according to paragraph 26, wherein the formulation comprises: 10 to 200 mg/ml, such as 10 to 140 mg/ml of the IL-13R antibody or binding fragment thereof (for example 10, 15, 20, 25, 30, 35, 40, 45, 50, 55, 60, 65, 70, 75, 80, 85, 90, 95, 100, 105, 110, 115, 120, 125, 130, 135 or 140 mg/ml (or 145, 150, 155, 160, 165, 170, 175, 180, 185, 190, 195, or 200 mg/ml));
   50 to 200 mM or arginine, such as 50 mM to 150 mM of arginine (for example 50, 55, 60, 65, 70, 75, 80, 85, 90, 95, 100, 105, 110, 115, 120, 125, 130, 135, 140, 145 or 150 mM arginine, (or 155, 160, 165, 170, 175, 180, 185, 190, 195, or 200 mM, such as 100 mM arginine)); 15 to 25 mM histidine buffer, for example 15, 16, 17, 18, 19, 20, 21, 22, 23, 24 and 25, such as 20 mM histidine buffer;
   0.01-0.03% of a non-ionic surfactant, such as 0.02% w/v and
   wherein the pH of the formulation is in the range 5.5 to 7.5 for example 6.2 to 7.2 (such as 6.2, 6.3, 6.4, 6.5, 6.6, 6.7, 6.8, 6.9, 7.0, 7.1, 7.2), such as 6.5 to 7.0, in particular 6.4 to 6.9).
28. The method according to any one of paragraphs 22 or 23, wherein the osmolarity of the formulation is in the range 250 to 550 mOsmo/kg, such as 350 to 550 mOsmo/kg, for example (250, 255, 260, 265, 270, 275, 280, 285, 290, 295, 300, 305, 310, 315, 320, 325, 330, 335, 340, 345 mOsmo/kg) 350, 355, 365, 370, 375, 380, 385, 390, 395, 400, 405, 410, 415, 420, 425, 430, 435, 440, 445, 450, 455, 460, 465, 470, 475, 480, 485, 490, 495, 500, 505, 515, 520, 525, 530, 535, 540, 545, 550, such as 405 to 435 mOsmo/kg.
29. The method according to any one of paragraphs 22 to 24, wherein the formulation further comprises 50 to 200 mM of a sugar, for example 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, such as 180 mM sugar.
30. The method according to any one of paragraphs 22 to 25, wherein the pH is 6.2 to 6.8, for example 6.2, 6.3, 6.4, 6.5, 6.6, 6.7 or 6.8, in particular 6.5.
31. The method according to any one of paragraphs 22 to 26, wherein the formulation does not comprise NaCl.
32. The method according to any one of paragraphs 22 to 27, wherein the formulation comprises 50 to 150 mM of NaCl, for example 50, 55, 60, 65, 70, 75, 80, 85, 90, 95, 100, 105, 110, 115, 120, 125, 130, 135, 140, 145, 150, such as 62.5 or 140 mM NaCl.
33. The method according to anyone of paragraphs 1 to 23, wherein the method is for the treatment or prophylaxis of an inflammatory disorder (such as chronic inflammation) or an autoimmune disease.
34. The method according to paragraph 29, wherein the inflammatory disorder or autoimmune disease is selected from the group comprising: fibrosis (including pulmonary fibrosis, such as cystic fibrosis, iodiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis (including seasonal rhinitis), asthma, chronic pulmonary disease (including chronic obstructive pulmonary disease), and allergy (for example a peanut allergy), in particular asthma.
35. The method according to paragraphs 29 or 30, wherein the inflammatory disorder is atopic dermatitis.
36. An anti-IL13R antibody or binding fragment (for example an anti-IL13R antibody or binding fragment thereof as defined in any one of paragraphs 10 to 15) for use in the treatment of an inflammatory disorder or an autoimmune disease, wherein each dose of the antibody or binding fragment thereof is in the range of about 1 mg/kg to about 15 mg/kg (about 60 to about 900 mg), for example about 1 mg/kg to about 10 mg/kg (about 60 to about 600 mg), about 3 mg/kg to 10 mg/kg (about 200 to about 600 mg), or about 10 mg/kg to about 15 mg/kg (about 600 to 900 mg), such as 3 mg/kg to 10 mg/kg (200 to 600 mg); and wherein each dose is administered at least once a month (4 weeks), for example once every 3 weeks, once every 2 weeks, once a week, in particular once a month.
37. Use of an anti-IL13R antibody or binding fragment thereof (for example an anti-IL13R antibody or binding fragment thereof as defined in any one of paragraphs 10 to 15) in the manufacture of a medicament for the treatment of an inflammatory disorder or an autoimmune disease, wherein each dose or unit dose of the antibody or binding fragment thereof is in the range of about 1 mg/kg to about 15 mg/kg (about 60 to about 900 mg), for example about 1 mg/kg to about 10 mg/kg (about 60 to about 600 mg), about 3 mg/kg to 10 mg/kg (about 200 to about 600 mg), or about 10 mg/kg to about 15 mg/kg (about 600 to 900 mg), such as 3 mg/kg to 10 mg/kg (200 to 600 mg); and wherein each dose or unit dose is administered at least once a month (4 weeks), for example once every 3 weeks, once every 2 weeks, once a week, or once daily, in particular once a month.

Thus, the present disclosure provides a method of treatment comprising inhibiting IL-13R with an antibody or binding fragment thereof specific for the receptor,

wherein each dose of the anti-IL13R antibody or binding fragment thereof is in the range of about 1 mg/kg to about 15 mg/kg (about 60 to about 900 mg), for example about 3 mg/kg to about 15 mg/kg (about 200 to about 900 mg), about 3 mg/kg to 10 mg/kg (about 200 to about 600 mg), or about 10 mg/kg to about 15 mg/kg (about 600 to about 900 mg), in particular about 3 mg/kg to about 10 mg/kg (about 200 to about 600 mg); and wherein each dose is administered intravenously at least once a month, for example once every 4 weeks, once every 3 weeks, once every 2 weeks, or once a week, in particular only once a month.

In one embodiment the antibody, binding fragment or formulation is administered once every two weeks.

In one embodiment the antibody, binding fragment or formulation is administered once every three weeks.

In one embodiment the antibody, binding fragment or formulation is administered 1 or less times a month, for example 1 administration per month or 1.5 administrations a month (i.e. three administrations over 2 months).

The present disclosure extends to an antibody, binding fragment or formulation for use in a treatment regimen described herein.

Advantageously, the presently disclosed method results in inhibition, such as complete inhibition of STAT6 signalling and complete IL-13 receptor occupancy for around 1 week (7 days) or more, such as 2 weeks, 3 weeks or 4 weeks (or one month).

In one embodiment inhibition of STAT6 is maintained (for example at a therapeutic level) for a period of 14, 15, 16, 17, 18, 19, 20, 21, 22, 23, 24, 25, 26, 27, 28, 29 or 30 days, such as 29 days.

In one embodiment the receptor bound by the antibody or binding fragment is fully occupied, for example for a period a of 14, 15, 16, 17, 18, 19, 20, 21, 22, 23, 24, 25, 26, 27, 28, 29 or 30 days, such as 29 days.

Thus, in one embodiment a pharmacodynamic (for example full pharmacodynamic) effect is provided for a period of at least 14, 15, 16, 17, 18, 19, 20, 21, 22, 23, 24, 25, 26, 27, 28, 29 or 30 days, such as 29 days.

Furthermore, after administration there is rapid onset of action, for example the onset of action is within 12 hours or less, such as 11, 10, 9, 8, 7, 6, 5, 4, 3 or 2 hours, more specifically 1 hour, in particular 1 hour after IV administration.

The present inventors have demonstrated that the inhibitory action of the presently claimed anti-IL13R antibody or binding fragment thereof is rapid, with complete inhibition achievable within 1 hour following administration (such as intravenous administration) of the antibody or binding fragment thereof.

Furthermore, the dosing regimen of the present disclosure may inhibit other allergic mediators, such as TARC (thymus and activated regulated chemokine).

In addition, the dosing regimen of the present disclosure may minimise side effects, for example reduced or eliminate incidences of conjunctivitis and/or have reduced reaction at the injection site. Thus, the present inventors have established that the presently disclosed dosage levels can be safely tolerated with no evidence of adverse side effects.

Further advantageously, the present inventors have established that the duration of IL-13R inhibition is closely associated with the dosage level. Specifically, by increasing the dosage, the duration of IL-13R inhibition can be increased, and by extension the frequency of dosing can be reduced. Accordingly, the claimed method can be specifically tailored according to treatment requirements.

In one embodiment the lowest concentration for a pharmacodynamic effect (such as a full pharmacodynamic effect) is in the range 0.5 to 70 mg/L, such as 50 to 70 mg/L, for example 50, 51, 52, 53, 54, 55, 56, 57, 58, 59, 60, 60, 60.5, 61, 61.5, 62, 63, 64, 65, 66, 67, 68, 69 or 70 mg/L, for example drug serum levels.

In one embodiment the lowest concentration for a pharmacodynamic effect (such as a full pharmacodynamic effect) is in the range 0.5 to 20 mg/L, such as 0.5, 1 2, 3, 4, 5, 6, 7, 8, 9, 10, 11, 12, 13, 14, 15, 16, 17, 18, 19 or 20 mg/L.

In one embodiment the lowest concentration for a pharmacodynamic effect (such as a full pharmacodynamic effect) is in the range 1 to 10 mg/L.

In one embodiment the lowest concentration for a pharmacodynamic effect (such as a full pharmacodynamic effect) is in the range 0.5 to 2.5 mg/L.

In one embodiment the drug serum levels between doses (trough levels) is in the range 0.5 to 20 mg/L, such as 0.5, 1 2, 3, 4, 5, 6, 7, 8, 9, 10, 11, 12, 13, 14, 15, 16, 17, 18, 19 or 20 mg/L.

In one embodiment the drug serum levels between doses (trough levels) is in the range 1 to 10 mg/L, In one embodiment the drug serum levels between doses (trough levels) is in the range 0.5 to 2.5 mg/L.

Thus, in one embodiment, the dose, dose frequency and route of administration is selected so as to maintain a drug serum level above from about 0.5 to 20 mg/L (such as 1 to 10 mg/L) between doses.

Thus, in one embodiment, the dose, dose frequency and route of administration is selected so as to maintain a drug plasma level above from about 0.5 to 20 mg/L (such as 1 to 10 mg/L) between doses.

Suitable routes of administration are intravenous and/or subcutaneous administration, and preferred dose frequencies are once per week, once per two weeks, once per three weeks, and once per four weeks.

In one embodiment the dose or doses is/are administered intravenously.

Thus, intravenous dosing according to the present disclosure may be once per week, once per two weeks, once per three weeks or once per four weeks.

In one embodiment the dose or doses is/are administered intravenously only once each week.

In one embodiment the dose or doses is/are administered intravenously only once every two weeks.

In one embodiment the dose or doses is/are administered intravenously only once every three weeks.

In one embodiment the dose or doses is/are administered intravenously only once each month.

In one embodiment the dose or doses is/are administered subcutaneously.

In one embodiment the dose or doses is/are administered subcutaneously only once each week.

In one embodiment the dose or doses is/are administered subcutaneously only once every two weeks.

In one embodiment the dose or doses is/are administered subcutaneously only once every three weeks.

In one embodiment the dose or doses is/are administered subcutaneously only once each month.

Subcutaneous dosing according to the present disclosure may be once per week, once per two weeks, once per three weeks or once per four weeks.

In one aspect, there is provided a method of treatment comprising inhibiting IL-13R with an antibody or binding fragment thereof specific for the receptor with a VH sequence of SEQ ID NO: 51 or a sequence at least 95% identical thereto, and VL sequence of SEQ ID NO: 53 or a sequence at least 95% identical thereto, wherein said antibody or binding fragment is administered at a dose in the range 200 mg to 900 mg intravenously only once each month.

In another aspect, there is provided a method of treatment comprising inhibiting IL-13R with an antibody or binding fragment thereof specific for the receptor with a VH sequence of SEQ ID NO: 51 or a sequence at least 95% identical thereto, and VL sequence of SEQ ID NO: 53 or a sequence at least 95% identical thereto, wherein said antibody or binding fragment is administered at a dose in the range 600 mg to 900 mg intravenously only once each month.

In one embodiment, each dose of the antibody or binding fragment thereof is in the range of about 1 mg/kg to about 15 mg/kg, for example 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5 or 15.0 mg/kg. This approximately corresponds to a dosage of about 60 mg to about 900 mg for an average adult of around 60 kg. Thus, in one embodiment, each dose is in the range of about 60 mg to 900 mg, for example 60, 70, 80, 90, 100, 150, 200, 250, 300, 350, 400, 450, 500, 550, 600, 650, 700, 750, 800, 850, 860, 870, 880 or 900 mg.

In one embodiment, each dose the antibody or binding fragment thereof is in the range of about 1 mg/kg to about 10 mg/kg, for example 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10.0 mg/kg. This approximately corresponds to a dosage of about 60 mg to about 600 mg for an adult Thus, in one embodiment, each dose is in the range of about 60 mg to 600 mg, for example 60, 70, 80, 90, 100, 150, 200, 250, 300, 350, 400, 450, 500, 550, 560, 570, 580, 590, or 600 mg.

In one embodiment, each dose of the antibody or binding fragment thereof is in the range of about 3 mg/kg to about 10 mg/kg, for example 3.0, 3.5, 4.0, 4.5, 5.0, 5.5, 6.0, 6.5, 7.0, 7.5, 8.0, 8.5, 9.0, 9.5 or 10.0 mg/kg. This approximately corresponds to a dosage of about 200 mg to about 600 mg for an adult Thus, in one embodiment, each dose is in the range of about 200 mg to 600 mg, for example 200, 210, 220, 230, 240, 250, 300, 350, 400, 450, 500, 550, 560, 570, 580, 590 or 600 mg.

In one embodiment, each dose of the antibody or binding fragment thereof is in the range of about 10 mg/kg to about 15 mg/kg, for example 10.0, 10.5, 11.0, 11.5, 12.0, 12.5, 13.0, 13.5, 14.0, 14.5 or 15.0 mg/kg. This approximately corresponds to a dosage of about 600 mg to about 900 mg for an adult Thus, in one embodiment, each dose is in the range of about 600 mg to 900 mg, for example 600, 610, 620, 630, 640, 650, 700, 750, 800, 850, 860, 870, 880 or 900 mg.

In one embodiment, each dose of the antibody or binding fragment thereof is about 1 mg/kg, for example 0.9, 0.95, 1.0, 1.05 or 1.1 mg/kg. This dose approximately corresponds to a dosage of about 60 mg for an adult Thus, in one embodiment, each dose is in the range of about 60 mg, such as 55, 56, 57, 58, 59, 60, 61, 62, 63, 64 or 65 mg. Advantageously, a dose of about 1 mg/kg expected to effectively inhibit IL-13R activity for about 7 days or 1 week. Thus, in one embodiment, each dose is administered once every 7 days or once a week.

In one embodiment, each dose of the antibody or binding fragment thereof is about 3.0 mg/kg, for example 2.5, 2.6, 2.7, 2.8, 2.9, 3.0, 3.1, 3.2, 3.3, 3.4 or 3.5 mg/kg. Advantageously, a dose of about 3.0 mg/kg expected to effectively inhibit IL-13R activity for about 21 days or 3 weeks. This dose approximately corresponds to a dosage of about 200 mg for an adult. In one embodiment, each dose of the anti-IL13R antibody or binding fragment thereof is about 200 mg, such as 190, 195, 200, 205 or 210 mg. Advantageously, a dose of about 200 mg is expected to effectively inhibit IL-13R activity for about 21 days or 3 weeks. Thus, in one embodiment, each dose is administered once every 3 weeks or every 21 days.

In one embodiment, each dose of the antibody or binding fragment thereof is about 10.0 mg/kg, for example 9.0, 9.5, 10.0, 10.5 or 11.0 mg/kg. Advantageously, a dose of about 10 mg/kg expected to effectively inhibit IL-13R activity for about 4 weeks or about one month. This dose approximately corresponds to a dosage of about 600 mg for an adult. In one embodiment, each dose of the anti-IL13R antibody or binding fragment thereof is about 600 mg, such as 590, 595, 600, 605 or 610 mg. Advantageously, a dose of about 600 mg is expected to effectively inhibit IL-13R activity for about a month or 4 weeks. Thus, in one embodiment, each dose is administered once every 4 weeks or once a month.

In one embodiment, each dose of the antibody or binding fragment thereof is about 15.0 mg/kg, for example 14, 14.5, 15.0 or 15.5 or 11.0 mg/kg. Advantageously, a dose of 15.0 mg/kg expected to effectively inhibit IL-13R activity for 4 weeks or longer. This dose approximately corresponds to a dosage of about 900 mg for an average adult. In one embodiment, each dose of the anti-IL13R antibody or binding fragment thereof is about 600 mg, such as 590, 595, 600, 605 or 610 mg. Advantageously, a dose of about 600 mg is expected to effectively inhibit IL-13R activity for about a month or 4 weeks. Thus, in one aspect, each dose is administered once every 4 weeks or once a month, or less, such as once every 5, 6, 7 or 8 weeks. In one aspect, each dose is administered every 5 weeks. In one aspect, each dose is administered every 6 weeks. In one aspect, each dose is administered every 7 weeks. In one embodiment, each dose is administered once every 8 weeks or every 2 months.

The dose frequency may range from about once very 7 days to about once every 4 weeks, i.e. about once a week to once a month.

Thus, in one embodiment, each dose of the anti-IL13R antibody or binding fragment thereof is administered every 7 days or once a week.

In one embodiment, each dose of the anti-IL13R antibody or binding fragment thereof is administered every 14 days or once every 2 weeks.

In one embodiment, each dose of the anti-IL13R antibody or binding fragment thereof is administered every 21 days or once every 3 weeks.

In one embodiment, each dose of the anti-IL13R antibody or binding fragment thereof is administered every 28 days or once every 4 weeks.

In one embodiment, each dose of the anti-IL13R antibody or binding fragment thereof is administered once a month, such as once every 28 days, once every 29 days, once every 30 days or once every 31 days.

In one embodiment, the dose is about 60 mg and is administered once every 7 days or once a week.

In one embodiment, the dose is about 200 mg and is administered once every 14 days or once every 2 weeks.

In one embodiment, the dose is about 600 mg and is administered once every 4 weeks or once a month.

In one embodiment, the dose is about 900 mg and is administered once a month or less, such as once every 5, 6, 7 or 8 weeks.

In one embodiment, the anti-IL-13R antibody or binding fragment is administered by infusion.

In one embodiment, the anti-IL-13R antibody or binding fragment is administered by infusion over a period of about 60 mins, such as 55, 56, 57, 58, 59, 60, 61, 62, 63, 64 or 65 mins.

In one embodiment the IL-13R antibody or binding fragment is administered via a syringe driver.

In one embodiment, the anti-IL-13R antibody or binding fragment is in the form of a pharmaceutical formulation, such as a parenteral formulation of the present disclosure.

In one embodiment, the anti-IL-13R antibody or binding fragment is ASLAN004 as disclosed herein.

Accordingly, in one embodiment, the antibody or binding fragment specific for IL-13R comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10; and a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.

In one embodiment, the antibody or binding fragment thereof comprises a VH domain comprising an amino acid sequence shown in SEQ ID NO: 51 or a sequence at least 95% identical thereto, and a VL domain comprising an amino acid sequence shown in SEQ ID NO: 53 or a sequence at least 95% identical thereto.

In one embodiment, the antibody or binding fragment specific for IL-13R comprises a VH sequence of SEQ ID NO: 51 and a VL sequence of SEQ ID NO: 53.

### DETAILED DISCLOSURE

One month as used herein refers to one calendar month, which includes all possible months in a year, including a leap year February which has 29 days. Thus, “once a month” may refer to once every 28 days, once every 29 days, once every 30 days or once every 31 days.

Unit dose as used herein generally refers to a product comprising the amount of anti-IL13R antibody or binding fragment thereof of the present disclosure that is administered in a single dose. A unit dose of the presently claimed anti-IL13R antibody or binding fragment thereof may refer to the marketed form of the product, such as a formulation of the anti-IL13R antibody or binding fragment thereof, wherein the product is apportioned into the precise amount of anti-IL13R antibody that is required for a single dose. Thus, the manufacturer is able to determine and control the exact amount of anti-13R antibody or binding fragment thereof to be included in each unit dose. The product may be in various forms, familiar to the skilled addressee, such as capsules, vials, tablets, patches, ampoules and the like, in particular vials.

Thus, a unit dose may be a single vial of anti-IL13R antibody formulation which contains the exact amount of anti-13R antibody that is needed for a single dose, whose entire contents may be directly administered to a patient without the need to first apportion out the required amount before administration.

Thus, in one embodiment, the dose is a unit dose. Accordingly, there is provided a unit dose of an anti-IL13R antibody or binding fragment thereof, wherein each unit dose of the anti-IL13R antibody or binding fragment thereof is in the range of about 1 mg/kg to about 15 mg/kg (about 60 to about 900 mg), for example about 1 mg/kg to about 10 mg/kg (about 60 to about 600 mg), about 3 mg/kg to 10 mg/kg (about 200 to about 600 mg), or about 10 mg/kg to about 15 mg/kg (about 600 to about 900 mg), in particular about 3 mg/kg to about 10 mg/kg (about 200 to about 600 mg).

In one embodiment, the unit dose is 600 mg to 900 mg, such as 600, 650, 700, 800, 850 or 900 mg.

In one embodiment, the formulation is a parenteral formulation.

Parenteral formulation as employed herein refers to a formulation designed not to be delivered through the GI tract. Typical parenteral delivery routes include injection (including bolus injection), implantation or infusion. In one embodiment the formulation is provided in a form for bolus delivery.

In one embodiment the parenteral formulation is administered intravenously, for example 50, 60, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 800, 805, 810, 815, 820, 825, 830, 835, 840, 845, 850, 855, 860, 865, 870, 875, 880, 885, 890, 900, 905, 910, 915, 920, 925, 930, 935, 940, 945, 950, 955, 960, 965, 970, 975, 980, 985, 990, 995 or 1000 mg of the anti-IL13R antibody or binding fragment thereof, in particular administered once a month.

In one embodiment the parenteral formulation is administered subcutaneously, for example 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 800, 805, 810, 815, 820, 825, 830, 835, 840, 845, 850, 855, 860, 865, 870, 875, 880, 885, 890, 900, 905, 910, 915, 920, 925, 930, 935, 940, 945, 950, 955, 960, 965, 970, 975, 980, 985, 990, 995, 1000, 1025, 1050, 1075, 1100, 1125, 1150, 1175, 1200, 1225, 1250, 1275, 1300, 1325, 1350, 1375, 1400, 1425, 1450, 1475, 1500 mg of the anti-IL13R antibody or binding fragment thereof, in particular once a month.

In one embodiment the subcutaneous dose of the anti-IL13R antibody or binding fragment thereof is in the range 200 mg to 1000 mg.

In one embodiment the parenteral formulation is administered intramuscularly, for example 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 800, 805, 810, 815, 820, 825, 830, 835, 840, 845, 850, 855, 860, 865, 870, 875, 880, 885, 890, 900, 905, 910, 915, 920, 925, 930, 935, 940, 945, 950, 955, 960, 965, 970, 975, 980, 985, 990, 995, 1000, 1025, 1050, 1075, 1100, 1125, 1150, 1175, 1200, 1225, 1250, 1275, 1300, 1325, 1350, 1375, 1400, 1425, 1450, 1475, 1500 mg of the anti-IL13R antibody or binding fragment thereof, in particular once a month.

In one embodiment the parenteral formulation is a depot formulation, for example administered with a dose of 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 800, 805, 810, 815, 820, 825, 830, 835, 840, 845, 850, 855, 860, 865, 870, 875, 880, 885, 890, 900, 905, 910, 915, 920, 925, 930, 935, 940, 945, 950, 955, 960, 965, 970, 975, 980, 985, 990, 995, 1000, 1025, 1050, 1075, 1100, 1125, 1150, 1175, 1200, 1225, 1250, 1275, 1300, 1325, 1350, 1375, 1400, 1425, 1450, 1475, 1500 mg of the anti-IL13R antibody or binding fragment thereof, in particular once a month.

In one embodiment the dose of the anti-IL13R antibody or binding fragment thereof is 600 mg or more.

In one embodiment the dose of the anti-IL13R antibody or binding fragment thereof is 8 to 10 mg/Kg.

Injection as employed herein refers to the administration of a liquid formulation into the body via a syringe or syringe driver. Injection includes intravenous, subcutaneous, intra-tumoral or intramuscular administration. The injection is generally over a short period of time, such as 5 minutes or less. However, injection can be administered slowly or continuously, for example using a syringe driver. Injections generally involve administration of smaller volumes than infusions. In one embodiment the injection is administered as a slow injection, for example over a period of 1.5 to 30 minutes. Slow injection as employed herein is manual injection with syringe.

Injections are usually smaller volumes than infusions, for example 30 mLs or less will usually be considered an injection.

In one embodiment one dose of the formulation less than 100 mls, for example 30 mls, such as administered by a syringe driver.

Infusion as employed herein means the administration of fluids by drip, infusion pump, or equivalent device. In one embodiment the infusion is administered over a period in the range of 1 to 120 minutes (for example 1 to 5 minutes), such as about 1, 3, 4, 5, 6, 7, 8, 9, 10, 11, 12, 13, 14, 15, 16, 17, 18, 19 20, 25, 30, 35, 40, 45, 50, 55, 60, 65, 70, 65, 80, 85, 90, 95, 100, 105, 110, 115 or 120 minutes. In one embodiment, the infusion is administered over a period of about 60 mins, such as 50, 51, 52, 53, 54, 55, 56, 57, 58, 59, 60, 61, 62, 63, 64, 65, 66, 67, 68, 69 or 70 mins, in particular over 60 mins.

Infusion usually involves administration of larger volumes than injections, for example the volume will generally be more than 30 mL.

Bolus injection as employed herein refers to the administration of a large amount of formulation in a single “shot”. This may be administered intravenously, intramuscularly or subcutaneously. It may be formulated for slow release, for example as a depot injection.

Depot formulation as employed herein refers to formulations which has an increased residence time in vivo (also referred to as injectable modified release product), which provides slow release of the active agent (the antibody or binding fragment). Generally the depot formulation will be for subcutaneous or intramuscular administration.

Examples of depot formulations include where the antibody or binding fragment is PEGylated or modified to comprise a further binding domain which binds serum albumin.

Formulations such as these may also be administered intravenously, as the skilled person is aware.

Other types of depot formulations include providing the antibody or binding fragment in an oil, such as sesame seed oil.

Protamine may be employed in depot formulations.

Polymer carriers may be employed in depot formulations, for example PLA, PLGA, PLGA-glucose, PLGA formulated with N-methyl-2-pyrollidone, PLGA polyesters (such as Eligard®, Atridox®, H.P. Acthar Gel), gelatin, amino acid polymers, DL-lactic and glycolic acid copolymer, Atrigel™, and polylactide/glycolide formulations.

Liposomes may be employed in depot formulations, including lipid nanoparticles coated with PEG.

Anti-IL13R antibody Interleukin-13 receptor (IL-13R) as used herein is a cytokine receptor, which binds to Interleukin-13. It consists of two subunits: IL13Rα1 and IL4R, respectively. These subunits form a dimer. IL-13 binds to the IL-13Rα1 chain and IL4 binds to the IL-4Rα chain. Therefore, IL13R can also instigate IL-4 signalling. In both cases signalling occurs via activation of the Janus kinase (JAK)/Signal Transducer and Activator of Transcription (STAT) pathway, resulting in phosphorylation of STAT6. Human IL-13Rα1 has the Uniprot number P3597.

IL-13Rα2, previously called IL-13R and IL-13Rα, is another receptor which is able to bind to IL-13. However, in contrast to IL-13Rα1, this protein binds IL-13 with high affinity, but it does not bind IL-4. Human IL-13Rα2 has the Uniprot number Q14627.

Anti-IL13R antibody as used herein refers to an antibody that has specificity for IL13R, for example IL13Rα1 or IL13Rα2.

In one embodiment, the anti-IL13R antibody of the present disclosure is specific for IL13Rα1. In one embodiment, the anti-IL13R antibody binds to an epitope comprising the amino acid sequence FFYQ.

The anti-IL13R antibodies of the present disclosure may comprise a complete antibody molecule having full length heavy and light chains or a binding fragment thereof. Binding fragments include but are not limited to Fab, modified Fab, Fab′, F(ab′)₂, Fv, single domain antibodies (such as VH, VL, VHH, IgNAR V domains), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217).

The methods for creating and manufacturing these antibody fragments are well known in the art (see for example Verma et al, 1998, Journal of Immunological Methods, 216, 165-181). Other antibody fragments for use in the present invention include the Fab and Fab′ fragments described in WO2005/003169, WO2005/003170 and WO2005/003171. Other antibody fragments for use in the present invention include Fab-Fv and Fab-dsFv fragments described in WO2010/035012 and antibody fragments comprising those fragments. Multi-valent antibodies may comprise multiple specificities or may be monospecific (see for example WO 92/22853 and WO05/113605).

The antibody and fragments thereof, for use in the present disclosure may be from any species including for example mouse, rat, shark, rabbit, pig, hamster, camel, llama, goat or human. Chimeric antibodies have a non-human variable regions and human constant regions.

An antibody or binding fragment for use in the present invention can be derived from any class (e.g. IgG, IgE, IgM, IgD or IgA) or subclass of immunoglobulin molecule. In one embodiment the antibody employed in the present disclosure is IgG4 or IgG4 with a hinge stabilising S241P (Kabat numbering) mutation.

In one embodiment the antibody or binding fragment employed in the formulation of the present disclosure has affinity of 5 nM or higher (higher affinity is a lower numerical value), for example 500 pM, such as 250 pM or higher affinity, in particular 125 pM or a lower numerical value.

(SEQ ID NO: 1)
In one embodiment CDRH1 comprises an amino acid
sequence GYSFTSYWIG.
(SEQ ID NO: 2)
In one embodiment CDRH2 comprises a sequence
VIYPGDSYTR

In one embodiment CDRH3 comprises the formula:

SEQ ID NO: 3
X₁ Pro Asn Trp Gly X₆ X₇ Asp X₉
X₁ denotes Phe, Met, Gln, Leu or Val
X₆ denotes Ser or Ala
X₇ denotes Phe, Leu, Ala or Met
X₉ denotes Tyr, Gln, Lys, Arg, Trp, His, Ala, Thr,
Ser, Asn or Gly

In one embodiment the IL13-R1α1 antibody or binding fragment employed in the formulation of the present disclosure comprises a CDRH3 independently selected from a sequence comprising SEQ ID NO: 4 to 30 in the sequence listing filed herewith. These sequences are also shown in Table 1 of the priority document, which is specifically incorporated herein by reference In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: or 3.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 5, 6, 7, 8, 9, 10, 11, 12, 13, 14, 15, 16, 17, 18, 19, 20, 21, 22, 23, 24, 25, 26, 27, 28, 29 or 30.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.

(SEQ ID NO: 31)
In one embodiment CDRL1 is a sequence comprising
RASQSISSSYLA.
(SEQ ID NO: 32)
In one embodiment CDRL2 is a sequence comprising
GASSRAT.

In one embodiment CDL3 comprises the formula:

SEQ ID NO: 33
Gln X₂X₃X₄X₅
X₂ denotes Gln, Arg, Met, Ser, Thr or Val.
X₃ denotes Tyr or Val.
X₄ denotes Glu, Ala, Gly or Ser.
X₅ denotes Thr, Ala or Ser.

In one embodiment the IL-13Rα1 antibody employed in the formulation of the present disclosure comprises a CDRL3 independently selected from a sequence comprising SEQ ID NO: 34 to 47 in the sequence listing filed herewith. These sequences are also shown in Table 2 of the priority document, which is specifically incorporated herein by reference.

In one embodiment, the anti-IL-13Rα antibody or binding fragment employed in the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33.

In one embodiment, the anti-IL-13Rα antibody of the present disclosure comprises a VL CDR1 comprising an amino acid sequence SEQ ID NO: 84, a VL CDR2 comprising an amino acid sequence SEQ ID NO: 85, and a VL CDR3 comprising an amino acid sequence as set forth in SEQ ID NO: 34 35, 36, 37, 38, 39, 40, 41, 42, 43, 44, 45, 46, or 47.

In one embodiment, the anti-IL-13Rα antibody of the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: or 3, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 34, 35, 36, 37, 38, 39, 40, 41, 42, 43, 44, 45, 46, or 47.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment the VH region is independently selected from a sequence from the group comprising: SEQ ID NO: 48; SEQ ID NO: 49; SEQ ID NO: 50; SEQ ID NO: 51 and a sequence at least 95% identical to any one of the same.

In one embodiment the VL is independently selected from a sequence from the group comprising:

SEQ ID NO: 52
EIVLTQSPGTLSLSPGERATLSCRASQSISSSYLAWYQQKPGQAPRLLIY
GASSRATGIPDRFSGSGSGTDFTLTISRLEPEDFAVYYCQQYETFGQGTK
VEI*
SEQ ID NO: 53
EIVLTQSPGTLSLSPGERATLSCRASQSISSSYLAWYQQKPGQAPRLLIY
GASSRATGIPDRFSGSGSGTDFTLTISRLEPEDFAVYYCQQYASFGQGTK
VEI*
SEQ ID NO: 54
EIVLTQSPGTLSLSPGERATLSCRASQSISSSYLAWYQQKPGQAPRLLIY
GASSRATGIPDRFSGSGSGTDFTLTISRLEPEDFAVYYCQQYEAFGQGTK
VEI*
SEQ ID NO: 55
(null sequence)

and a sequence at least 95% identical to any one of the same (* K deleted in a post translational modification).

In one embodiment the VH sequence is SEQ ID NO: 48 (or a sequence at least 95% identical thereto) and the VL sequence is SEQ ID NO: 52, SEQ ID NO: 53 or SEQ ID NO: 54 (or a sequence at least 95% identical to any one of the same).

In one embodiment the VH sequence is SEQ ID NO: 49 (or a sequence at least 95% identical thereto) and the VL sequence is SEQ ID NO: 52, SEQ ID NO: 53 or SEQ ID NO: 54 (or a sequence at least 95% identical to any one of the same).

In one embodiment the VH sequence is SEQ ID NO: 50 (or a sequence at least 95% identical thereto) and the VL sequence is SEQ ID NO: 52, SEQ ID NO: 53 or SEQ ID NO: 54 (or a sequence at least 95% identical to any one of the same).

In one embodiment the VH sequence is SEQ ID NO: 51 (or a sequence at least 95% identical thereto) and the VL sequence is SEQ ID NO: 52, SEQ ID NO: 53 or SEQ ID NO: 54 (or a sequence at least 95% identical to any one of the same).

In one embodiment the VL sequence is SEQ ID NO: 52 (or a sequence at least 95% identical thereto) and the VH sequence is SEQ ID NO: 48, SEQ ID NO: 49, SEQ ID NO: 50 or SEQ ID NO: 51. (or a sequence at least 95% identical to any one of the same) In one embodiment the VL sequence is SEQ ID NO: 53 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VL sequence is SEQ ID NO: 54 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VH sequence is SEQ ID NO: 51 (or a sequence at least 95% identical thereto) and the VL sequence is SEQ ID NO: 53 ((or a sequence at least 95% identical thereto).

Variable region as employed herein refers to the region in an antibody chain comprising the CDRs and a suitable framework.

In one embodiment the heavy chain comprises a sequence independently selected from the group comprising:

SEQ ID NO: 56
EVQLVQSGAEVKKPGESLKISCKGSGYSFTSYWIGWVRQMPGKGLEWMGV
IYPGDSYTRYSPSFQGQVTISADKSISTAYLQWSSLKASDTAMYYCARMP
NWGSFDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLG*
SEQ ID NO: 57
EVQLVQSGAEVKKPGESLKISCKGSGYSFTSYWIGWVRQMPGKGLEWMGV
IYPGDSYTRYSPSFQGQVTISADKSISTAYLQWSSLKASDTAMYYCVRMP
NWGSLDHWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLG*
SEQ ID NO: 58
EVQLVQSGAEVKKPGESLKISCKGSGYSFTSYWIGWVRQMPGKGLEWMGV
IYPGDSYTRYSPSFQGQVTISADKSISTAYLQWSSLKASDTAMYYCVRMP
NWGSLDHWGQGTLVTVSSASIKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLG*
SEQ ID NO: 59
EVQLVQSGAEVKKPGESLKISCKGSGYSFTSYWIGWVRQMPGKGLEWMGV
IYPGDSYTRYSPSFQGQVTISADKSISTAYLQWSSLKASDTAMYYCARMP
NWGSLDHWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLG*
SEQ ID NO: 60
EVQLVQSGAEVKKPGESLKISCKGSGYSFTSYWIGWVRQMPGKGLEWMGV
IYPGDSYTRYSPSFQGQVTISADKSISTAYLQWSSLKASDTAMYYCARMP
NWGSLDHWGQGTLVTVSSASIKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLG*
SEQ ID NO: 61
EVQLVQSGAEVKKPGESLKISCKGSGYSFTSYWIGWVRQMPGKGLEWMGV
IYPGDSYTRYSPSFQGQVTISADKSISTAYLQWSSLKASDTAMYYCARMP
NWGSLDHWGQGTLVTVSSASTKGPSVFPLAPCSRSTSESTAALGCLVKDY
FPEPVTVSWNSGALTSGVHTFPAVLQSSGLYSLSSVVTVTSSNFGTQTYT
CNVDHKPSNTKVDKTVERKCCVECPPCPAPPVAGPSVFLFPPKPKDTLMI
SRTPEVTCVVVDVSQEDPEVQFNWYVDGVEVHNAKTKPREEQFNSTFRVV
SVLTVLHQDWLNGKEYKCKVSNKGLPSSIEKTISKTKGQPREPQVYTLPP
SREEMTKNQVSLTCLVKGFYPSDIAVEWESNGQPENNYKTTPPMLDSDGS
FFLYSKLTVDKSRWQQGNVFSCSVMHEALHNHYTQKSLSLSPG*,

and a sequence at least 95% identical to any one of the same (*K deleted in a post translational modification) In one embodiment the light chain is independently selected from a group comprising: SEQ ID NO: 62: SEQ ID NO: 63; SEQ ID NO: 55 and a sequence at least 95% identical to any one of the same.

In one embodiment the heavy chain is independently selected from SEQ ID NO: 56, 57, 58, 59, 60 and 61 (or a sequence at least 95% identical to any one of the same) and the light chain is independently selected from SEQ ID NO: 62, 63 and 55 (or a sequence at least 95% identical to any one of the same).

In one embodiment the heavy chain is SEQ ID NO: 56 (or a sequence at least 95% identical thereto) and the light chain is independently selected from SEQ ID NO: 62, 63 and 55 (or a sequence at least 95% identical to any one of the same).

In one embodiment the heavy chain is SEQ ID NO: 57 (or a sequence at least 95% identical thereto) and the light chain is independently selected from SEQ ID NO: 62, 63 and 55 (or a sequence at least 95% identical to any one of the same).

In one embodiment the heavy chain is SEQ ID NO: 58 (or a sequence at least 95% identical thereto) and the light chain is independently selected from SEQ ID NO: 62, 63 and 55 (or a sequence at least 95% identical to any one of the same).

In one embodiment the heavy chain is SEQ ID NO: 59 (or a sequence at least 95% identical thereto) and the light chain is independently selected from SEQ ID NO: 62, 63 and 55 (or a sequence at least 95% identical to any one of the same).

In one embodiment the heavy chain is SEQ ID NO: 60 (or a sequence at least 95% identical thereto) and the light chain is independently selected from SEQ ID NO: 62, 63 and 55 (or a sequence at least 95% identical to any one of the same).

In one embodiment the heavy chain is SEQ ID NO: 61 (or a sequence at least 95% identical thereto) and the light chain is independently selected from SEQ ID NO: 62, 63 and 55 (or a sequence at least 95% identical to any one of the same).

In one embodiment the heavy chain is SEQ ID NO: 59 or 61 (or a sequence at least 95% identical to any one of the same) and a light chain with the sequence shown in SEQ ID NO: 62 (or a sequence at least 95% identical thereto).

In one embodiment the heavy chain is SEQ ID NO: 59 (or a sequence at least 95% identical to any one of the same) and a light chain with the sequence shown in SEQ ID NO: 62 (or a sequence at least 95% identical thereto).

In one embodiment the heavy chain is SEQ ID NO: 61 (or a sequence at least 95% identical to any one of the same) and a light chain with the sequence shown in SEQ ID NO: 62 (or a sequence at least 95% identical thereto).

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

Any discussion of a protein, antibody or amino acid sequence herein will be understood to include any variants of the protein, antibody or amino acid sequence produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” (C-terminal lysine residues of encoded antibodies are often removed during the manufacturing process) and/or have part or all of a signal sequence incompletely processed and, as a consequence, remain at the terminus of the antibody. It is understood that an antibody comprising a particular amino acid sequence or binding fragment thereof may be a heterogeneous mixture of the stated or encoded sequence and/or variants of that stated or encoded sequence or binding fragment thereof.

In one embodiment the present disclosure extends to a sequence explicitly disclosed herein where the C-terminal lysine has been cleaved.

In one embodiment an antibody or binding fragment thereof, employed in a formulation of the present disclosure is humanised.

Humanised (which include CDR-grafted antibodies) as employed herein refers to molecules having one or more complementarity determining regions (CDRs) from a non-human species and a framework region from a human immunoglobulin molecule (see, for example U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al.). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody employed in the present invention, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

In one embodiment the anti-IL13R antibodies of the present disclosure are fully human, in particular one or more of the variable domains are fully human.

Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP0438474 and EP0463151.

Constant region as employed herein is intended to refer to the constant region portion located between two variable domains, for example non-cognate variable domains, in the heavy chain. Thus, the presently disclosed anti-IL13R antibody may comprise one or more constant regions, such as a naturally occurring constant domain or a derivate of a naturally occurring domain.

A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present disclosure may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO93/06231, WO92/22583, WO89/00195, WO89/01476 and WO03/031581. Alternatively, where the effector molecule is a protein or polypeptide the linkage may be achieved using recombinant DNA procedures, for example as described in WO86/01533 and EP0392745.

The term effector molecule as used herein includes, for example, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful, for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (e.g. U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

In one embodiment the antibody or binding fragment employed in the formulation of the present disclosure is monoclonal.

In one embodiment the antibody or binding fragment employed in the formulation of the present disclosure is human.

In one embodiment the antibody or binding fragment employed in the formulation of the present disclosure is chimeric or humanised.

### Treatment

Less than twice a month as employed herein refers to the average of doses over at least a two-month period, for example 3 doses in two months is on average 1.5 doses per month. However, in practice it will mean administration of one dose in one month and two doses in the next month.

The anti-IL13R antibody or binding fragment thereof or formulation thereof according to the present disclosure may be used for treatment or in the manufacture of a medicament. For example, the disclosed anti anti-IL13R antibody or binding fragment thereof or formulation thereof is suitable for use in treating an inflammatory disorder, such as chronic inflammation, or an autoimmune disease.

The inflammatory condition or disorder, may, for example be selected from the group comprising or consisting of arthritis such as rheumatoid arthritis, asthma such as severe asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease, transplant rejection, heart disease including ischaemic diseases (such as myocardial infarction as well as atherosclerosis), intravascular coagulation, bone resorption, osteoporosis, osteoarthritis, periodontitis, hypochlorhydia and cancer, including breast cancer, lung cancer, gastric cancer, ovarian cancer, hepatocellular cancer, colon cancer, pancreatic cancer, esophageal cancer, head & neck cancer, kidney, and cancer, in particular renal cell carcinoma, prostate cancer, liver cancer, melanoma, sarcoma, myeloma, neuroblastoma, placental choriocarcinoma, cervical cancer, and thyroid cancer, and the metastatic forms thereof.

In one embodiment the autoimmune disease is selected from the group comprising or consisting of Acute disseminated encephalomyelitis (adem), acute necrotizing hemorrhagic leukoencephalitis, Addison's disease, adrenal insufficiency, hypocortisolism, alopecia areata, amyloidosis, ankylosing spondylitis, spondyloarthritis, Strumpell-marie disease, anti-GBM/anti-TBM nephritis, antiphospholipid syndrome (aps), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune lymphoproliferative syndrome (ALPS), Canale-Smith syndrome, autoimmune myocarditis, autoimmune oophoritis, autoimmune pancreatitis (AIP), autoimmune polyglandular syndromes (types I, II & III), autoimmune retinopathy (AR), autoimmune thrombocytopenic purpura (ATP), autoimmune thyroid disease, autoimmune urticaria, axonal/neuronal neuropathies, balo disease, Behcet's disease, bullous pemphigoid, cardiomyopathy, Castleman disease, coeliac disease, chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid (CP), Crohn's disease, inflammatory bowel disease, colitis, enteritis, ileitis, Cogans syndrome, cold agglutinin disease, congenital heart block, Coxsackie myocarditis, crest disease, cryoglobulinemia, demyelinating neuropathies, dermatitis herpetiformis, Duhring's disease, dermatomyositis, diabetes, type I, discoid lupus erythematosus (DLE), Dressler's syndrome, endometriosis, epidermolysis bullosa (EB) and eb acquisita (EBA), eosinophilic gastroenteritis, esophagitis, eosinophilic fasciitis, schulman's syndrome, erythema nodosum, experimental allergic encephalomyelitis, Evans syndrome, fibrosing alveolitis, giant cell arteritis (temporal arteritis), giant cell myocarditis, glomerulonephritis (non-proliferative: focal segmental glomerulosclerosis and membranous glomerulonephritis. proliferative: IgA nephropathy), goodpasture's syndrome, granulomatosis with polyangiitis (GPA) (formerly called Wegener's granulomatosis), Graves' disease, Guillain-Barre syndrome, Miller Fisher syndrome, acute motor axonal neuropathy, acute motor sensory axonal neuropathy, acute panautonomic neuropathy, Bickerstaff's brainstem encephalitis, Hashimoto's encephalitis, Hashimoto's thyroiditis, hemolytic anemia, Henoch-Schonlein purpura, herpes gestationis, hypogammaglobulinemia, idiopathic pulmonary fibrosis, idiopathic thrombocytopenic purpura (ITP), IgA nephropathy (IGAN), berger's syndrome, synpharyngitic glomerulonephritis, IgA pemphigus, IgG4-related sclerosing disease, immune-regulated infertility, inclusion body myositis, insulin-dependent diabetes mellitus, interstitial cystitis, Isaac's syndrome, neuromyotonia, juvenile arthritis, juvenile myositis, Kawasaki syndrome, Lambert-Eaton syndrome, leukocytoclastic vasculitis, lichen planus, lichen sclerosus, ligneous conjunctivitis, linear IgA dermatosis (LAD), pemphigoid, lupus (SLE), lyme disease, Meniere's disease, microscopic polyangiitis (MPA), mixed connective tissue disease (MCTD), monoclonal gammaopathy, Mooren's ulcer, Mucha-Habermann disease, multiple sclerosis, myasthenia gravis, myositis, narcolepsy, neuromyelitis optica (devic's), neuromyotonia, Isaac's syndrome (acquired, paraneoplastic, hereditary), neutropenia, ocular cicatricial pemphigoid, optic neuritis, oophoritis, opsoclonus-myoclonus syndrome, orchitis, palindromic rheumatism, pandas (pediatric autoimmune neuropsychiatric disorders associated with Streptococcus), paraneoplastic autoimmune multiorgan syndrome (PAMS), paraneoplastic cerebellar degeneration, paraneoplastic pemphigus (PNP), paroxysmal nocturnal hemoglobinuria (PNH), Parry Romberg syndrome, Parsonnage-Turner syndrome, pars planitis (peripheral uveitis), pempgigoid gestationis (PG), pemphigus vulgaris (PV), pemphigus folliaceus (PF), peripheral neuropathy, perivenous encephalomyelitis, pernicious anemia, Poems syndrome, polyarteritis nodosa (PAN), polymyalgia rheumatic, polymyositis, postmyocardial infarction syndrome, postpericardiotomy syndrome, progesterone dermatitis primary biliary cirrhosis, Hanot syndrome, primary sclerosing cholangitis (PSC), sclerosong cholangitis, psoriasis, psoriatic arthritis, pyoderma gangrenosum, pure red cell aplasia, Rasmussen's encephalitis, chronic focal encephalitis (CFE), Raynauds phenomenon, reactive arthritis, Reiter's syndrome, recoverin-associated retinopathy (RAR), reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, systemic sclerosis, Sjogren's syndrome, sperm & testicular autoimmunity, stiff person/man syndrome, subacute bacterial endocarditis (SBE), Susac's syndrome, sympathetic ophthalmia, Takayasu's arteritis, temporal arteritis/giant cell arteritis, thromboangiitis obliterans, Buerger's disease, thrombocytopenic purpura (TTP), Tolosa-Hunt syndrome, transverse myelitis, ulcerative colitis, undifferentiated connective tissue disease (UCTD), uveitis, polymyalgia rheumatica, Takayasu's arteritis, temporal arteritis, Buerger's disease, cutaneous vasculitis, Kawasaki disease, polyarteritis nodosa, Behçet's syndrome, Churg-Strauss syndrome, cutaneous vasculitis, Henoch-Schönlein purpura, microscopic polyangiitis, Wegener's granulomatosis, golfer's vasculitis, vesiculobullous dermatosis, and Vitiligowegener's granulomatosis (now termed granulomatosis with polyangiitis (GPA).

In one embodiment the autoimmune disease is selected from the group comprising or consisting of ANCA vasculitis, IgA nephropathy (Berger's), pemphigus vulgaris/bullous pemphigoid, ITP, primary biliary cirrhosis, autoimmune thyroiditis (Grave's disease), hashimoto's disease, lupus nephritis, membranous glomerulonephritis (or membranous nephropathy), APS, myasthenia gravis, neuromyelitis optica, primary Sjögren's, autoimmune neutropaenia, autoimmune pancreatitis, dermatosmyositis, autoimmune uveitis, autoimmune retinopathy, Behçet's disease, IPF, systemic sclerosis, liver fibrosis, autoimmune hepatitis, primary sclerosing cholangitis, vitiligo, goodpasture's syndrome, pulmonary alveolar proteinosis, chronic autoimmune urticarial, psoriasis, rheumatoid arthritis, psoriatic arthritis, axial spodyloarthritis, transplantation (including GvHD), asthma, COPD, giant cell arteritis, refractory autoimmune cytopaenias, Evans syndrome (autoimmune haemolytic anaemia), type I diabetes, sarcoidosis, polymyositis, ulcerative colitis, Crohn's disease, coeliac disease, Waldenstrom's macroglobulinaemia, focal segmental glomerulosclerosis, chronic Lyme disease (Lyme borreliosis), lichen planus, Stiff person syndrome, dilated cardiomyopathy, autoimmune (lymphocytic) oophoritis, epidermolysis bullosa acquisita, autoimmune atrophic gastritis, pernicious anaemia, atopic dermatitis, atherosclerosis, multiple sclerosis, Rasmussen's encephalitis, Guillain-Barre syndrome, acquired neuromyotonia, stroke.

In one embodiment the antibody or antigen-binding fragment thereof or formulation, according to the present disclosure is employed for the treatment of a chronic inflammatory condition wherein the condition associated with inappropriate inflammation. Such conditions include, but are not limited to, rheumatoid arthritis (RA), autoimmune conditions, inflammatory bowel diseases, non-healing wounds, multiple sclerosis, cancer, atherosclerosis, sjogrens disease, diabetes, lupus erythrematosus (including systemic lupus erythrematosus), asthma, fibrotic diseases (including liver cirrhosis), pulmonary fibrosis, and UV damage and psoriasis.

Chronic inflammation is a debilitating and serious condition associated with many of the above diseases and is characterised by persistent inflammation at a site of infection or injury, or persistent inflammation of an unknown origin, or in relation to altered immune responses such as in autoimmune disease.

Thus, in one embodiment the antibody or antigen-binding fragment, formulation or method according to the present disclosure is employed in the treatment of a chronic inflammatory condition wherein the condition is associated with any condition associated with inappropriate inflammation. Such conditions include, but are not limited to, rheumatoid arthritis (RA), autoimmune conditions, inflammatory bowel diseases, non-healing wounds, multiple sclerosis, cancer, atherosclerosis, Sjogrens disease, diabetes, lupus erythrematosus (including systemic lupus erythrematosus), asthma, fibrotic diseases (including liver cirrhosis), pulmonary fibrosis, UV damage and psoriasis.

In one embodiment the antibody or antigen-binding fragment thereof, formulation or method according to the present disclosure is employed in the treatment of a condition selected from axial spondyloarthropathy, primary biliary cholangitis, and allergy, for example a food allergy such as a peanut allergy, or a pollen allergy.

In one embodiment the inflammatory disorder or autoimmune disease is selected from the group comprising: fibrosis (including pulmonary fibrosis, such as cystic fibrosis, iodiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis, asthma and chronic pulmonary disease (including chronic obstructive pulmonary disease).

In patients with cancer, such as breast cancer, cancer related lymphedema (BCRL), the formulation of the present disclosure may prevent lymphedema-associated effects, such as fibrosis, hyperkeratosis, the deposition of fibroadipose tissue, fluid accumulation, limb swelling, reduction of skin elasticity, and pain. By reducing the excess volume, said formulation may improve lymphatic and, for example limb functions.

The development of lymphedema after lymphatic injury is associated with tissue inflammation, the infiltration of CD4-positive cells and their differentiation to the type 2 helper T-cell (Th2) phenotype. Th2 cells produce IL-4 and IL-13 that play a key role in the development of lymphedema-associated symptoms as well as other Th2-mediated diseases.

In one embodiment the antibody, binding fragment or formulation of the present disclosure is used for the treatment of asthma or is used for the manufacture of a medicament for the treatment of the same.

In one embodiment the antibody, binding fragment or formulation of the present disclosure is used for the treatment of dermatitis (such as atopic dermatitis) or is used for the manufacture of a medicament for the treatment of the same.

In one embodiment the antibody, binding fragment or formulation of the present disclosure is used for the treatment of Psoriasis or is used for the manufacture of a medicament for the treatment of the same.

In one embodiment the antibody, binding fragment or formulation of the present disclosure is employed as a monotherapy.

In one embodiment the formulation herein is administered in combination with another therapy, for example an anti-inflammatory agent, such as a non-steroidal anti-inflammatory and/or a steroid (eg prednisolone or prednisolone).

“In combination” as employed herein is intended to encompass where the anti-IL13R antibody is administered before, concurrently with another therapy.

Therapeutic dose as employed herein refers to the amount of the anti-IL13R antibody, such as ASLAN004 that is suitable for achieving the intended therapeutic effect when employed in a suitable treatment regimen, for example ameliorates symptoms or conditions of a disease, in particular without eliciting dose limiting side effects. Suitable therapeutic doses are generally a balance between therapeutic effect and tolerable toxicity, for example where the side-effect and toxicity are tolerable given the benefit achieved by the therapy.

In one embodiment a formulation according to the present disclosure (including a formulation comprising same) is administered monthly, for example in a treatment cycle or as maintenance therapy.

### Formulations of Anti-IL-13R Antibodies

Antibodies, such as ASLAN004 need to be formulated to high concentration to allow the desired dose in man to be administered in the smallest possible volume. High concentration formulations pose unique challenges as phenomena like phase separation can be observed. Aggregation is also a common feature at high antibody concentration. However, the formulation needs to contain very high levels of antibody molecules as “monomer”, for example 95% monomer or more. In addition, the formulation needs to be stable when stored. ASLAN004 seem to have a hydrophobic portion in the protein, which for example interacts with hydrophobic interaction columns in the absence of high salt concentrations. This hypothesised hydrophobic portion adds additional complexity when formulating the antibody and preventing aggregation. Thus, the antibodies of the present disclosure are particularly difficult to formulate.

The present inventors have optimised the formulation of the present disclosure and established that the IL-13R antibodies, such as ASLAN004, are most suitable for formulation within a narrow set of parameters. The formulations of the present disclosure are highly monomeric, for example at least 95% monomeric (such as 98 to 99.5% monomeric) even when formulated with high antibody concentration. In addition, the formulation is suitably stable, for example in some embodiments no change in monomer or less than a 0.5% reduction in monomer was observed when stored at 4° C. or 25° for 90 days. Accelerated ‘stress test’ studies at 40° C. also show the formulations of the present disclosure to be stable over a period of 60 days, for example using potency measurements.

The combination of features of the formulation of the present disclosure, including the pH, contributing to stabilising the IL-13 receptor antibody or binding fragment thereof.

In one embodiment the formulations of the present disclosure has a viscosity in the range of 4.5 to 5.5, such as 4.5, 4.6, 4.7, 4.8, 4.9, 5.0, 5.1, 5.2, 5.3, 5.4 or 5.5 cP (centipoise), such as 4.9 cP, for example at ambient temperature. Surprisingly, the viscosity of the formations of the present disclosure are relatively low even at high concentrations of antibody.

In one embodiment the osmolarity of the formulation is in the range 350 to 450 mOsmo/kg, such as 390 to 430 mOsmo/kg, in particular 410+/−5 mOsmo/kg.

In one embodiment, the formulation further comprises 10 to 145 mg/ml anti-IL13R antibody, for example 10 to 125 mg/ml, such as 10, 20, 30, 40, 50, 60, 70, 80, 90, 100, 110 or 120 mg/ml, in particular 20 mg/ml or 100 mg/ml of anti-IL13R antibody.

In one embodiment certain formulations of the present disclosure have 5% or less protein aggregation, such 4, 3, 2, 1% or less, for example when stored for 90 days at temperature in the range 2 to 25° C.

The presently disclosed anti-IL13R antibody formulation is particularly suitable for stable long-term storage of the anti-IL13R antibody.

Long term as used herein refers to a period of at least 6 months, such as 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months. In one embodiment, the disclosed formulation storage for at least 12 months, such as 12 months, 18 months and 24 months.

In one embodiment the formulation is stored at a temperature in the range 2 to 8° C., such as 2, 3, 4, 5, 6, 7 or 8° C., such as 4° C.

In one embodiment there is provided a parenteral formulation (in particular a liquid formulation) for example for infusion or injection. In one embodiment there is provided liquid parenteral formulation as a concentrate for dilution with a liquid for injection, such as glucose, saline or water for injection. In one embodiment the liquid parenteral formulation is provided in a final concentration for administration without dilution, for example for injection or for infusion.

In one embodiment, arginine is L-arginine.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

Subject headings herein are employed to divide the document into sections and are not intended to be used to construe the meaning of the disclosure provided herein.

This specification claims priority from SG10201902713S filed 26 Mar. 2019, SG10201905063R filed 3 Jun. 2019, and SG10201907597W filed 16 Aug. 2019. Each of these are incorporated by reference, in particular the sequences and Figures. The priority documents may be used as basis for corrections to the present specification.

The present invention is further described by way of illustration only in the following examples.

### BRIEF SUMMARY OF THE FIGURES

FIG. 1 Shows an IgE assay for a 3 mg/Kg IV dose

FIG. 2 Shows the results of the pSTAT6 and RO Assays when 0.1 mg/kg ASLAN004 is administered intravenously.

FIG. 3 Shows the results of the pSTAT6 and RO Assays when 0.3 mg/kg ASLAN004 is administered intravenously.

FIG. 4 Shows the results of the pSTAT6 and RO Assays when 1 mg/kg ASLAN004 is administered intravenously. S5021 D85 RO data point was excluded due to assay error. D15 for S5016 and S5017 was tested on D12. D85 for S5021 was tested on D82.

FIG. 5 Shows the results of the pSTAT6 and RO Assays when 3.0 mg/kg ASLAN004 is administered intravenously. D15 for S5032 was tested on D12.

FIG. 6 Shows the results of the pSTAT6 and RO Assays when 10.0 mg/kg ASLAN004 is administered intravenously.

FIG. 7 Shows ASLAN004 SAD PK data-IV (serum levels measured).

FIG. 8 Shows the results of the pSTAT6 and RO Assays when 75 mg/kg ASLAN004 is administered subcutaneously.

FIG. 9 Shows the results of the pSTAT6 and RO Assays when 150 mg/kg ASLAN004 is administered subcutaneously.

FIG. 10 Shows the results of the pSTAT6 and RO Assays when 300 mg/kg ASLAN004 is administered subcutaneously.

FIG. 11 Shows the results of the pSTAT6 and RO Assays when 600 mg/kg ASLAN004 is administered subcutaneously.

FIG. 12 Shows a comparison of the ASLAN004 PK data with the Duplilumab PK data (A) intravenous (B) subcutaneous (serum levels measured).

FIG. 13 Shows a schematic representation of a potential theory behind the lower C_{trough} for ASLAN004 compared to Duplilumab.

### ABBREVIATIONS

pSTAT6—Signal transducer and activator of transcription 6
RO—Receptor occupancy
IV—intravenous
SC—subcutaneous
SAD—Single ascending dose

### AE—Adverse Event

### PD—Pharmacodynamic

### EXAMPLES

### ASLAN004 Formulation

2 formulations of ASLAN004 were prepared: a 20 mg/ml ASLAN004 formulation and a 100 mg/ml ASLAN004 formulation. Each formulation comprises 20 mM Histidine-HCl pH 6.5, 180 mM Sucrose, 100 mM Arginine, and 0.02% polysorbate 20.

### Single Ascending Dose (SAD) Study

Healthy volunteers were administered a single dose of the ASLAN004 formulation over a 60-minute intravenous infusion (IV) via a syringe driver or via a subcutaneous injection (SC). The following cohorts were conducted:

TABLE 1
Cohorts in SAD study

| | | | | Number of |
|---|---|---|---|---|
| Cohort | | | Mode of | volunteers |
| number | Dose of ASLAN004 given | | administration | tested |
| 1 | 0.1 | mg/kg | IV | 2 |
| 2 | 0.3 | mg/kg | IV | 3 |
| 3 | 1.0 | mg/kg | IV | 3 |
| 4 | 3.0 | mg/kg | IV | 6 |
| 5 | 10.0 | mg/kg | IV | 6 |
| 6* | 20.0 | mg/kg | IV | 6 |
| 7 | 75.0 | mg | SC | 6 |
| 8 | 150.0 | mg | SC | 6 |
| 9 | 300.0 | mg | SC | 6 |
| 10 | 600.0 | mg | SC | 6 |

*Cohort 6 was not actioned because a long PD effect of >29 days was achieved at 10 mg/kg.

The subcutaneous (SC) cohorts 7 to 10 were conducted in parallel after intravenous (IV) cohort 3 was completed.

Safety assessments included adverse events (AEs), vital signs and other clinical laboratory parameters. Serial blood samples were drawn for assessment of PK and PD parameters. Samples were taken pre-dose, 1 hour after dose, 24 hours after dose, 1 week after dose (Day 8), 2 weeks after dose (Day 15), 4 weeks after dose (Day 29) and 12 weeks after dose (Day 85). IgE levels were measured and pSTAT6 and RO assays were conducted.

### IgE Test

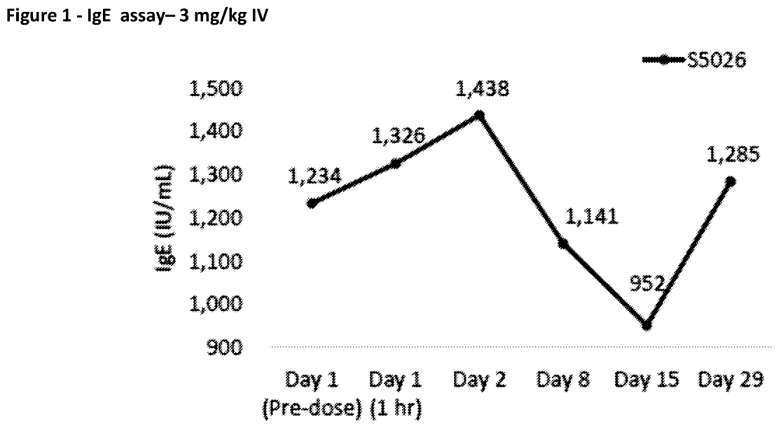

FIG. 1 shows a sample result for a volunteer who was given the 3 mg/kg IV dose. As a reference point, the normal expected IgE range is 0 to 87 IU/ml. As can be seen from FIG. 1, ASLAN004 resulted in an approximately 34% reduction in IgE levels, with the lowest levels of IgE measured on Day 15 (2 weeks after dose). The PD effect was lost around Day 29 (4 weeks after dose).

Accordingly, the results demonstrate the efficacy of ASLAN004 in suppressing IgE levels and suggests its potential for treating inflammation disorders.

### Adverse Event (AE) Profile

No AEs were observed that could be directly attributed to ASLAN004. No injection site reactions were observed, with only one case of mild itch that resolved within 24 hours. The volunteers had a common phase I AE profile. There was also no conjunctivitis or dry eye reported. This is in stark contrast with patients treated with duplilumab, with around 10% of patients suffering this side effect according to the prescribing label and as high as 25 to 50% according to recent literature reports. Hence, the results indicate that ASLAN004 is safe and well tolerated and avoids the side effects seen in patients treated with duplilumab.

### SAD Pharmacokinetics and Pharmacodynamics

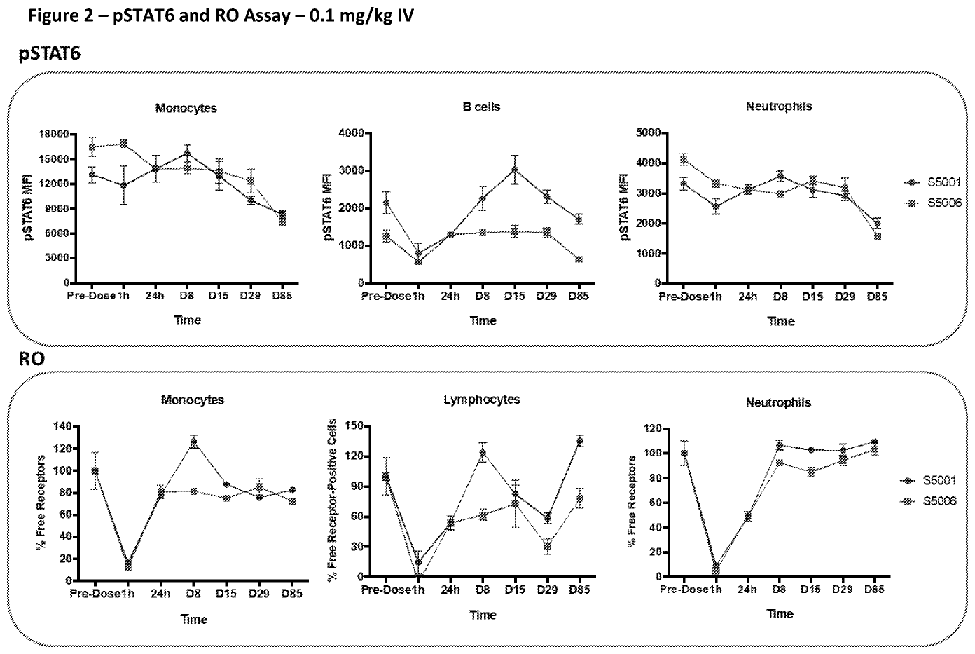

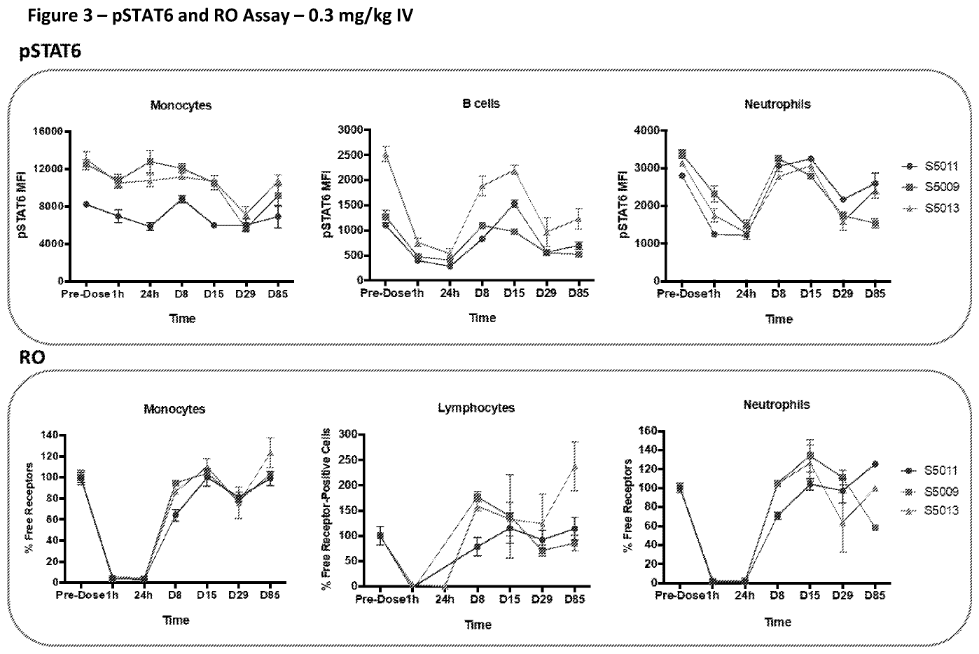

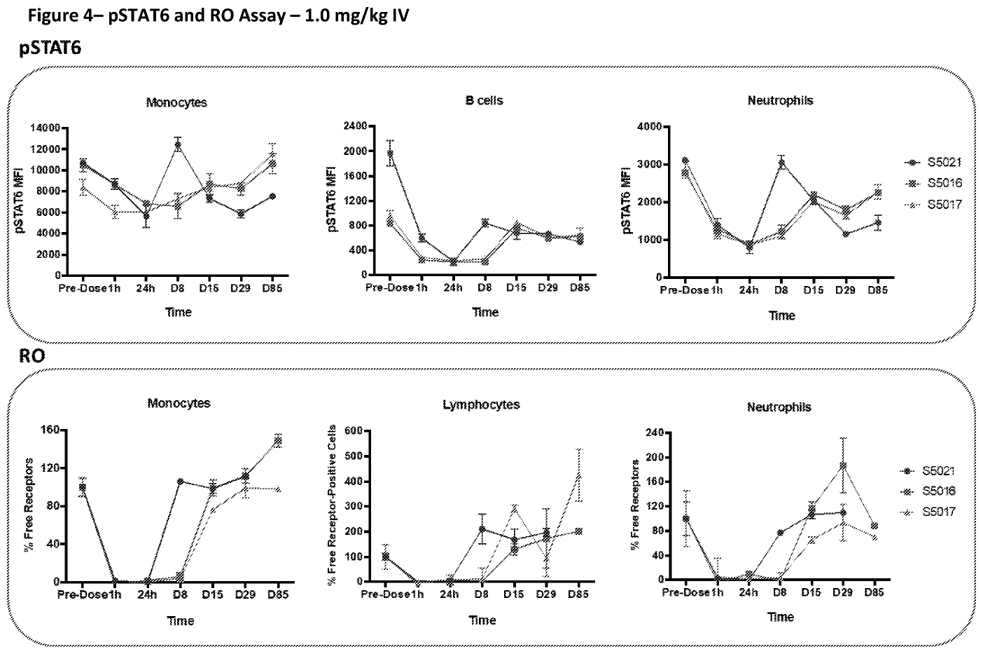

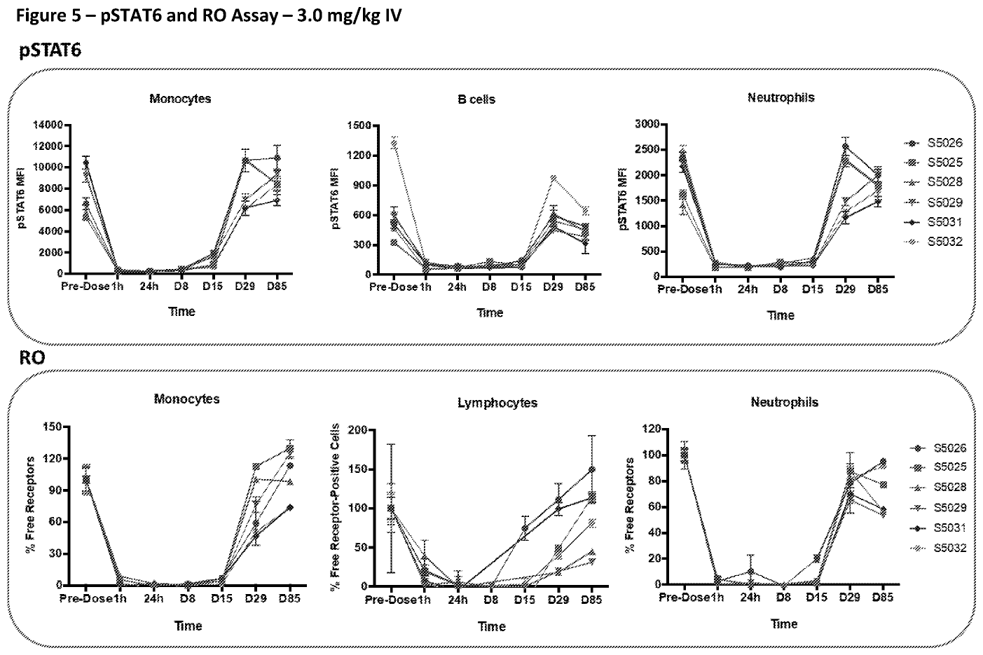

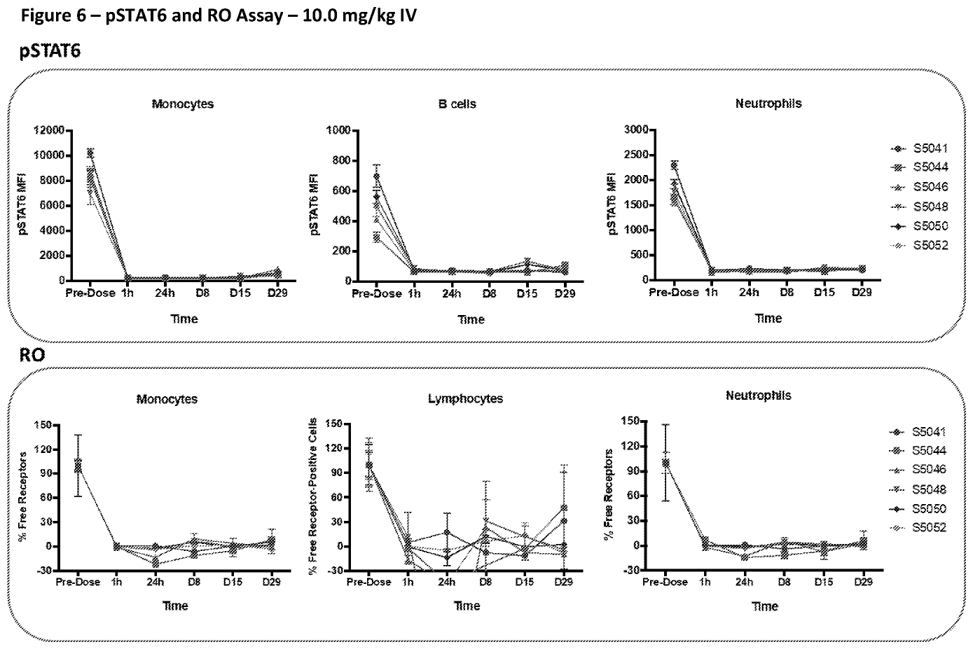

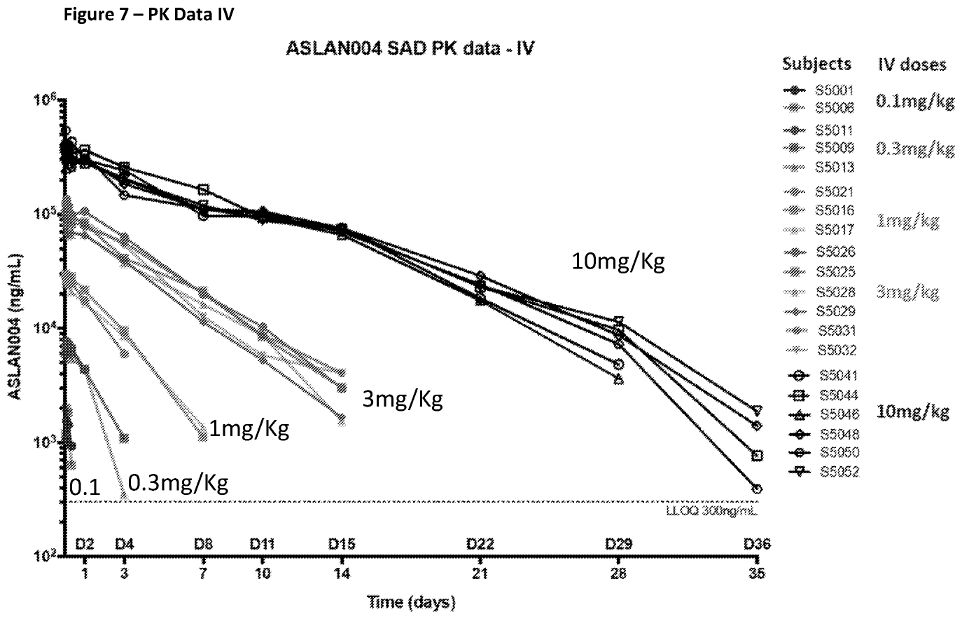

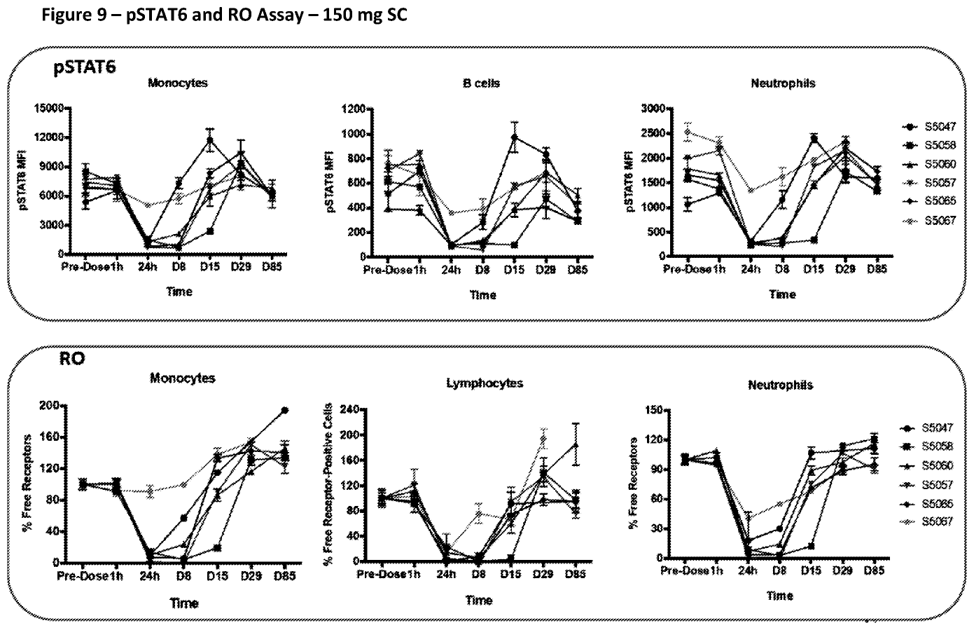

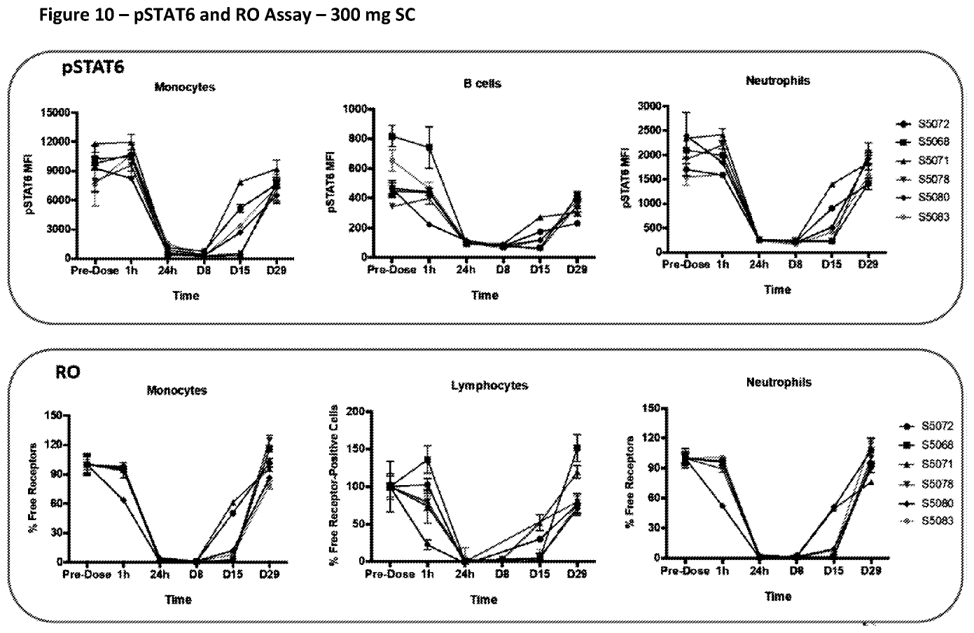

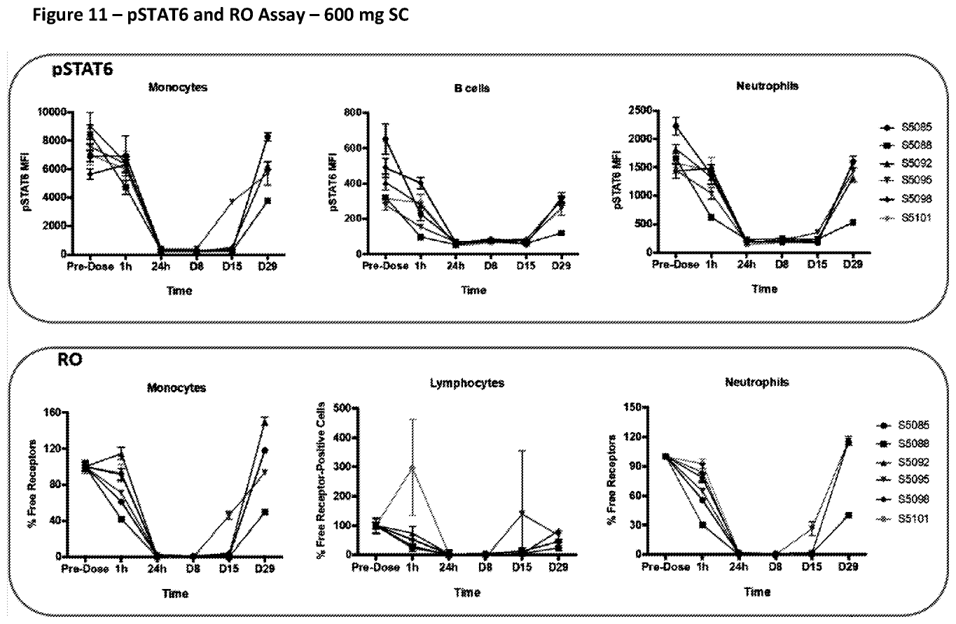

The results of the pSTAT6 and RO assays are shown in FIGS. 2 to 11. The results for the intravenous (IV) cohorts (FIGS. 2 to 6) suggest that the 0.1 mg/kg dose was able to achieve almost total receptor occupancy within 1 hour of administration of ASLAN004. However, this effect was not sustained and pSTAT6 and % free receptor levels started to rise shortly thereafter. The 0.3 mg/kg dose performed slightly better, achieving complete receptor inhibition, which lasted for about 24 hours. However, pSTAT6 and % free receptor levels again steadily rise after this.

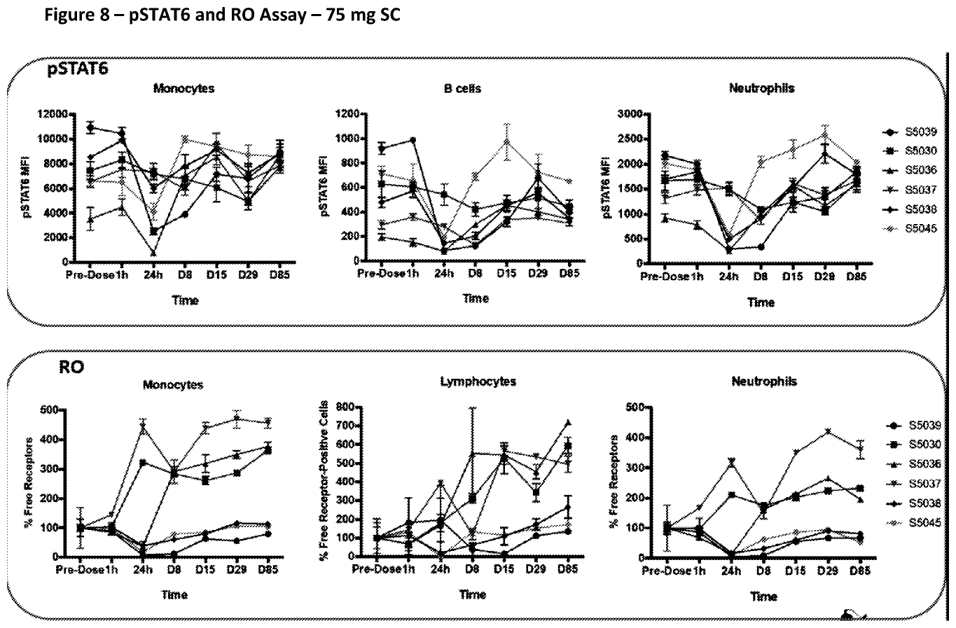

In contrast, at the 1 mg/kg dosage level, a sustained inhibition of pSTAT6 and % free receptor levels was observed for about 1 week (Day 8) following treatment with ASLAN004. Raising the dosage to 3 mg/kg further extended this effect to about 2 weeks (Day 15). This general trend continued with the 10 mg/kg dosage level wherein complete inhibition was achieved for around 4 weeks (Day 29). For the subcutaneous (SC) cohorts (FIGS. 8 to 11), the results suggest that the 75 mg dose was able to achieve almost total receptor occupancy within 24 hour of administration of ASLAN004. However, this effect was not sustained and pSTAT6 and % free receptor levels started to rise shortly thereafter.

However, at the 150 mg dosage level, a sustained inhibition of pSTAT6 and % free receptor levels was observed for about 1 week (Day 8) following treatment with ASLAN004. Raising the dosage to 300 mg further extended this effect to about 2 weeks (Day 15). A similar result was also observed for the 600 mg SC dose.

The table below shows the influence of subject weight on PD for subjects dosed with 600 mg SC:

TABLE 2
influence of subject weight on PD: 600 mg SC

| Subject | Weight (kg) | Full PD response to |
|---|---|---|
| S5085 | 70.6 | Day 15, PD lost by day 29 |
| S5088 | 65.3 | Day 15, partial PD to day 29 |
| S5092 | 76.3 | Day 15, PD lost by day 29 |
| S5095 | 82.3 | Day 8, partial PD to day 15 |
| S5098 | 76.3 | Day 15, PD lost by day 29 |
| S5101 | 68.8 | Day 15 |

These results may suggest that increasing subject weight negatively impacts on PD duration.

The following table summarises the PD details for the various doses tested:

TABLE 3
Summary table of PD details

| | | | | ASLAN004 concentration at |
|---|---|---|---|---|
| | | | Last timepoint of | last day of full PD effect |
| Dose | | Subject | full PD effect | (mg/L) |
| 0.1 | mg/kg | S5001 | 1 hour | 1.053 |
| | | S5006 | 1 hour | 1.186 |
| 1 | mg/kg | S5016 | Day 8 | 1.108 |
| | | S5017 | Day 8 | 1.352 |
| 3 | mg/kg | S5028 | Day 15 | 1.540 |
| | | S5029 | Day 15 | 1.658 |
| 75 | mg SC | S5045 | 24 hours | 0.556 |

| | | |
|---|---|---|
| S5039 | Day 8 | 1.162 |

| | |
|---|---|
| Average | 1.202 |

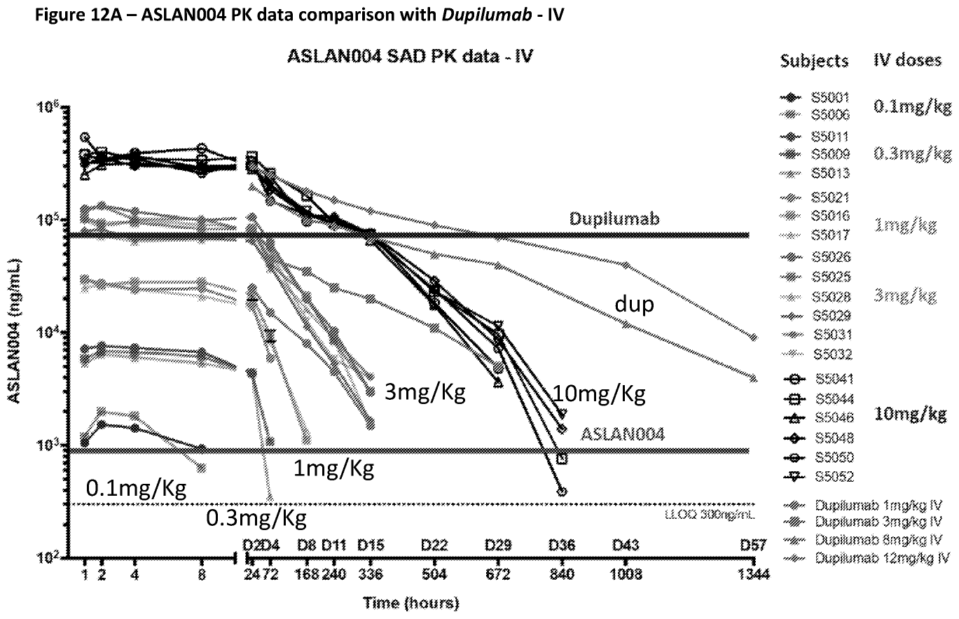

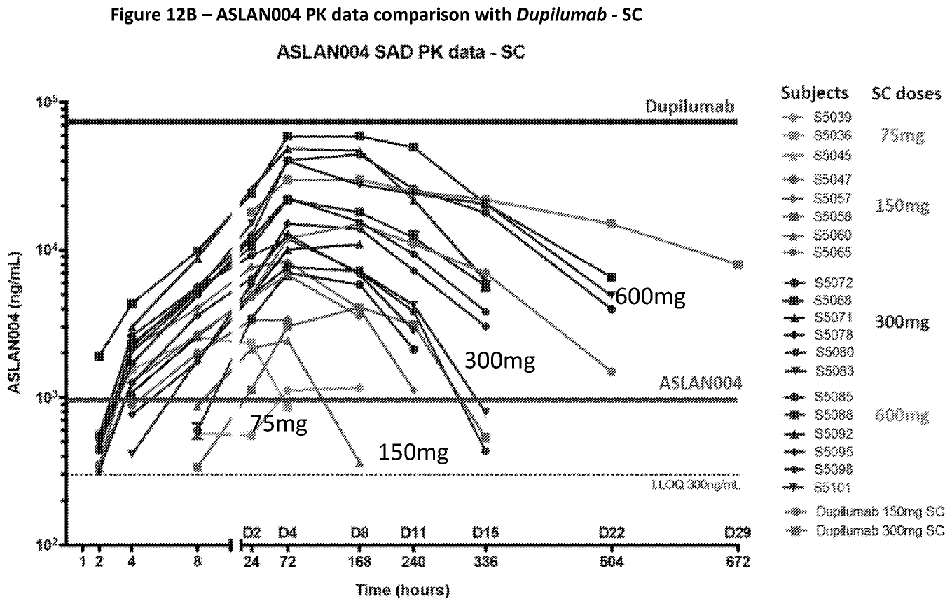

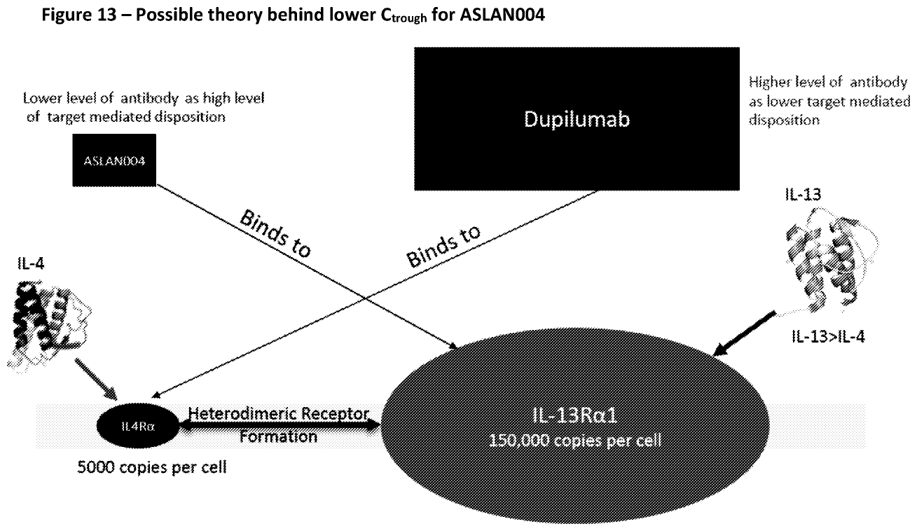

FIGS. 12A and 12B compare the PK data for ASLAN004 with the PK data for Dupilumab for IV and SC, respectively.

To summarise, the PK results suggest that ASLAN004 has a fast onset of action of less than 1 hour when administered intravenously (IV). In addition, the full PD effect (i.e. 100% binding to IL-13Rα1 and/or completely inhibition of pSTAT6 signaling) was achieved at approximately 1 mg/l. This full PD effect may be predicted to last for about a month with a dose of around 600 mg (i.e. 10 mg/kg) and an expected C_{trough} of 10 mg/l.

For comparison, Dupilumab has a C_{trough} level of 61.5 mg/l (based on week 16 data) and requires a bi-weekly dosage in order to provide full binding of IL-4Rα.

Without being bound to theory, the present inventors believe that the lower C_{trough} compared to Dupilumab for ASLAN004 can be achieved because ASLAN004 targets IL-13Rα1 and Dupilumab targets IL-4Rα. In vivo the numbers of IL-13Rα1 greatly outnumber the numbers of IL-4Rα. This means that a lower level of ASLAN004 antibody is required because of the higher level of target mediated deposition compared to Dupilumab.

Thus, the pharmacodynamic profile of ASLAN004 indicates that ASLAN004 compares very favourably to Duplilumab and suggests ASLAN004's the potential for monthly dosing to treat inflammatory disorders, such as atopic dermatitis

When greater than or equal to 600 mg ASLAN004 was administered intravenously (10 mg/kg) it demonstrated 100% receptor occupancy and complete inhibition of STAT6 phosphorylation in less than 1 hour after dosing. These effects were maintained for over 29 days following a single dose of ASLAN004, suggesting monthly dosing may be achievable. The rapid inhibition of IL-4 and IL-13 signaling by ASLAN004 could also lead to a fast onset of symptom relief in atopic dermatitis and allergic asthma patients.

1.-22. (canceled)

23. A pharmaceutical formulation for an anti-IL-13R antibody or binding fragment thereof, said formulation comprising:
10 to 200 mg/ml of the IL-13Rα1 antibody or binding fragment thereof;
50 to 200 mM of arginine;
15 to 25 mM histidine buffer;
0.01-0.03% of a non-ionic surfactant;
wherein the pH of the formulation is in the range 5.5 to 7.5.

24. The pharmaceutical formulation according to claim 23, wherein the IL-13Rα1 antibody or binding fragment thereof is in the range 20 to 140 mg/ml.

25. The pharmaceutical formulation according to claim 23 wherein the IL-13Rα1 antibody or binding fragment thereof is 100 mg/ml.

26. The pharmaceutical formulation according to claim 23, wherein the arginine is 100 mM.

27. The pharmaceutical formulation according to claim 23, wherein the histidine buffer is 20 mM.

28. A pharmaceutical formulation according to claim 23, wherein the non-ionic surfactant is 0.02%.

29. A pharmaceutical formulation according to claim 23, wherein the non-ionic surfactant is polysorbate 20.

30. A pharmaceutical formulation according to claim 23, wherein the pH is 6.5.

31. A pharmaceutical formulation according to claim 23, wherein the formulation further comprises 50 to 200 mM of a sugar.

32. A pharmaceutical formulation according to claim 31, wherein the sugar is 180 mM.

33. A pharmaceutical formulation according to claim 23, with a variable heavy region comprising a CDRH1 with a sequence shown in SEQ ID NO: 1; a CDRH2 with a sequence shown in SEQ ID NO: 3; and a variable light region comprising CDRL1 with a sequence shown in SEQ ID NO: 31, a CDRL2 with a sequence shown in SEQ ID NO: 32; and a CDRL3 with a sequence shown in SEQ ID NO: 33.

34. A pharmaceutical formulation according to claim 23

35. A method of treating an inflammatory disorder comprising administering a therapeutically effective amount of a pharmaceutical formulation for an anti-IL-13R antibody or binding fragment thereof, said formulation comprising:
10 to 200 mg/ml of the IL-13Rα1 antibody or binding fragment thereof;
50 to 200 mM of arginine;
15 to 25 mM histidine buffer;
0.01-0.03% of a non-ionic surfactant;
wherein the pH of the formulation is in the range 5.5 to 7.5.

36. The method according to claim 35, wherein the inflammatory response is atopic dermatitis.


SEQUENCE LISTING (64 sequences)

SEQ ID NO: 1 - protein, 10 aa, Artificial Sequence
  CDRH1
GYSFTSYWIG

SEQ ID NO: 2 - protein, 10 aa, Artificial Sequence
  CDRH2
VIYPGDSYTR

SEQ ID NO: 3 - protein, 9 aa, Artificial Sequence
  CDRH3
  MISC_FEATURE (1)..(1): Xaa denotes Phe, Met, Gln, Leu or Val
  MISC_FEATURE (6)..(6): Xaa denotes Ser or Ala
  MISC_FEATURE (7)..(7): Xaa denotes Phe, Leu, Ala or Met
  MISC_FEATURE (9)..(9): Xaa denotes Tyr, Gln, Lys, Arg, Trp, His, Ala, Thr, Ser, Asn or Gly
XPNWGXXDX

SEQ ID NO: 4 - protein, 9 aa, Artificial Sequence
  CDR H3 SEQUENCE
FPNWGALDQ

SEQ ID NO: 5 - protein, 9 aa, Artificial Sequence
  CDR H3 SEQUENCE
VPNMGSLDT

SEQ ID NO: 6 - protein, 9 aa, Artificial Sequence
  CDR H3 SEQUENCE
FPNWGSMDA

SEQ ID NO: 7 - protein, 9 aa, Artificial Sequence
  CDR H3 SEQUENCE
FPNWGSLDH

SEQ ID NO: 8 - protein, 9 aa, Artificial Sequence
  CDR H3 SEQUENCE
MPNWGSFDY

SEQ ID NO: 9 - protein, 9 aa, Artificial Sequence
  CDR H3 SEQUENCE
MPNWGSFDT

SEQ ID NO: 10 - protein, 9 aa, Artificial Sequence
  CDR H3 SEQUENCE
MPNWGSLDH

SEQ ID NO: 11 - protein, 9 aa, Artificial Sequence
  CDR H3 SEQUENCE
MPNWGSFDS

SEQ ID NO: 12 - protein, 9 aa, Artificial Sequence
  CDR H3 SEQUENCE
MPNWGSLDT

SEQ ID NO: 13 - protein, 9 aa, Artificial Sequence
  CDR H3 SEQUENCE
MPNWGSLDA

SEQ ID NO: 14 - protein, 9 aa, Artificial Sequence
  CDR H3 SEQUENCE
MPNWGSLDN

SEQ ID NO: 15 - protein, 9 aa, Artificial Sequence
  CDR H3 SEQUENCE
MPNWGALDS

SEQ ID NO: 16 - protein, 9 aa, Artificial Sequence
  CDR H3 SEQUENCE
MPNWGSLDN

SEQ ID NO: 17 - protein, 9 aa, Artificial Sequence
  CDR H3 SEQUENCE
MPNWGSLDY

SEQ ID NO: 18 - protein, 9 aa, Artificial Sequence
  CDR H3 SEQUENCE
MPNWGSFDH

SEQ ID NO: 19 - protein, 9 aa, Artificial Sequence
  CDR H3 SEQUENCE
MPNWGSLDS

SEQ ID NO: 20 - protein, 9 aa, Artificial Sequence
  CDR H3 SEQUENCE
MPNWGSLDG

SEQ ID NO: 21 - protein, 9 aa, Artificial Sequence
  CDR H3 SEQUENCE
VPNWGSLDG

SEQ ID NO: 22 - protein, 28 aa, Artificial Sequence
  CDR H3 SEQUENCE
CARFPNWGSLDHWGQGTLVTVSSASIKG

SEQ ID NO: 23 - protein, 28 aa, Artificial Sequence
  CDR H3 SEQUENCE
CARMPNWGSLDHWGQGTLVTVSSASTKG

SEQ ID NO: 24 - protein, 28 aa, Artificial Sequence
  CDR H3 SEQUENCE
CARMPNWGSFDYWGQGTLVTVSSASIKG

SEQ ID NO: 25 - protein, 12 aa, Artificial Sequence
  CDR H3 SEQUENCE
VRMPNWGSLDHW

SEQ ID NO: 26 - protein, 27 aa, Artificial Sequence
  CDR H3 SEQUENCE
VRMPNWGSLDHWGQGTLVTVSSADIKG

SEQ ID NO: 27 - protein, 27 aa, Artificial Sequence
  CDR H3 SEQUENCE
ARMPNWGSLDHWGQGTLVTVSSASIKG

SEQ ID NO: 28 - protein, 25 aa, Artificial Sequence
  CDR H3 SEQUENCE
FPNWGSFDYWGQGTLVTVSSASIKG

SEQ ID NO: 29 - protein, 9 aa, Artificial Sequence
  CDR H3 SEQUENCE
VPNWGSLDA

SEQ ID NO: 30 - protein, 9 aa, Artificial Sequence
  CDR H3 SEQUENCE
FPNWGSFDY

SEQ ID NO: 31 - protein, 12 aa, Artificial Sequence
  CDRL1
RASQSISSSYLA

SEQ ID NO: 32 - protein, 7 aa, Artificial Sequence
  CDRL2
GASSRAT

SEQ ID NO: 33 - protein, 5 aa, Artificial Sequence
  CDRL3
  MISC_FEATURE (2)..(2): Xaa denotes Gln, Arg, Met, Ser, Thr or Val
  MISC_FEATURE (3)..(3): Xaa denotes Tyr or Val
  MISC_FEATURE (4)..(4): Xaa denotes Glu, Ala, Gly or Ser
  MISC_FEATURE (5)..(5): Xaa denotes Thr, Ala or Ser
QXXXX

SEQ ID NO: 34 - protein, 5 aa, Artificial Sequence
  CDRL3 SEQUENCE
QRYAT

SEQ ID NO: 35 - protein, 5 aa, Artificial Sequence
  CDRL3 SEQUENCE
QRYST

SEQ ID NO: 36 - protein, 5 aa, Artificial Sequence
  CDRL3 SEQUENCE
QMYST

SEQ ID NO: 37 - protein, 5 aa, Artificial Sequence
  CDRL3 SEQUENCE
QQVGT

SEQ ID NO: 38 - protein, 5 aa, Artificial Sequence
  CDRL3 SEQUENCE
QQVST

SEQ ID NO: 39 - protein, 5 aa, Artificial Sequence
  CDRL3 SEQUENCE
QQYST

SEQ ID NO: 40 - protein, 5 aa, Artificial Sequence
  CDRL3 SEQUENCE
QSYST

SEQ ID NO: 41 - protein, 5 aa, Artificial Sequence
  CDRL3 SEQUENCE
QQYAT

SEQ ID NO: 42 - protein, 5 aa, Artificial Sequence
  CDRL3 SEQUENCE
QQYSS

SEQ ID NO: 43 - protein, 5 aa, Artificial Sequence
  CDRL3 SEQUENCE
QTYST

SEQ ID NO: 44 - protein, 5 aa, Artificial Sequence
  CDRL3 SEQUENCE
QQYGS

SEQ ID NO: 45 - protein, 5 aa, Artificial Sequence
  CDRL3 SEQUENCE
QQYAS

SEQ ID NO: 46 - protein, 5 aa, Artificial Sequence
  CDRL3 SEQUENCE
QQYEA

SEQ ID NO: 47 - protein, 5 aa, Artificial Sequence
  CDRL3 SEQUENCE
QQYET

SEQ ID NO: 48 - protein, 118 aa, Artificial Sequence
  VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 49 - protein, 118 aa, Artificial Sequence
  VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 50 - protein, 118 aa, Artificial Sequence
  VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 51 - protein, 118 aa, Artificial Sequence
  VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 52 - protein, 103 aa, Artificial Sequence
  VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 53 - protein, 103 aa, Artificial Sequence
  VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 54 - protein, 103 aa, Artificial Sequence
  VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 55 - protein, 211 aa, Artificial Sequence
  light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 444 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 443 aa, Artificial Sequence
  heavy chain
VQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 443 aa, Artificial Sequence
  heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 62 - protein, 211 aa, Artificial Sequence
  light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 211 aa, Artificial Sequence
  light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - skipped